(19) United States
(12) Patent Application Publication
(10) Pub. No.: US 2026/0014208 A1
(43) Pub. Date: Jan. 15, 2026

(54) DIFFERENTIATION INDUCER CONTAINING NUCLEUS PULPOSUS CELL MASTER REGULATOR TRANSCRIPTION FACTORS, METHOD FOR PRODUCING INDUCED NUCLEUS PULPOSUS CELLS, AND USE OF INDUCED NUCLEUS PULPOSUS CELLS

(71) Applicant: Tokai University Educational System, Tokyo (JP)

(72) Inventors: Daisuke SAKAI, Kanagawa (JP); Jordy SCHOL, Kanagawa (JP); Shunsuke HIRAISHI, Kanagawa (JP)

(73) Assignee: Kyoto University, Kyoto (JP)

(21) Appl. No.: 19/334,169

(22) Filed: Sep. 19, 2025

Related U.S. Application Data
(63) Continuation of application No. 17/599,276, filed on Sep. 28, 2021, now abandoned.

(30) Foreign Application Priority Data
Mar. 29, 2019 (JP) 2019-066535

(51) Int. Cl. A61K 35/32; A61K 38/00; A61K 49/00; C07K 16/32; C12N 5/077; C12N 15/86

(52) U.S. Cl. CPC A61K 35/32; A61K 49/0004; C07K 16/32; C12N 5/0655; C12N 15/86; A61K 38/00; C12N 2501/15; C12N 2501/19; C12N 2501/60; C12N 2506/13; C12N 2740/10043

(57) ABSTRACT

Provided is reproducible means that enables the production of an active nucleus pulposus cell phenotype from desired cells such as terminally differentiated cells or pluripotent or multipotent stem cells. Provided is a differentiation inducer containing an effective amount of a gene of at least two transcription factors selected from the group consisting of Brachyury (T), SRY-box6 (SOX6), and Forkhead Box Q1 (FOXQ1), or homologs thereof (nucleus pulposus cell master regulator transcription factor), or a product thereof.

### TECHNICAL FIELD

The present invention relates to transcription factors (nucleus pulposus cell master regulator transcription factors) that enable direct cell reconstruction (direct reprogramming) from terminally differentiated cells or cells with differentiation capacity to an active nucleus pulposus cell phenotype, that is, transdifferentiation or differentiation induction from undifferentiated cells to nucleus pulposus cells. The present invention further relates to an active nucleus pulposus cell phenotype (induced nucleus pulposus cells) obtained by transdifferentiation or differentiation induction, and to use thereof.

### BACKGROUND ART

Low back pain and neck pain are common health problems that affect 632 million people all over the world and a major cause of disability. The two disorders place significant social and economic burdens due to work disability and medical costs. Intervertebral disc degeneration, which is estimated to develop 20% of all low back pain cases, destructs biomechanics along the spinal column and leads to intervertebral hernia, spinal canal stenosis, spondylolisthesis, and other spinal disorders. Intervertebral disc degeneration is an intervertebral disc disorder that is particularly characterized by irreversible degeneration of the extracellular matrix composition in the core of an intervertebral disc.

At present, there is no clinically effective treatment that enables recovery from such a degenerative state or can stop the underlying pathogenesis. Therefore, there is a strong demand for the development of new treatments. Nevertheless, general understanding of intervertebral disc homeostasis, cell phenotype, and development and progression of the pathogenesis is poor, particularly as compared with knowledge insight in the field of bone and articular cartilage.

An intervertebral disc is the fibrocartilage structure between each two vertebrae along the spine. The intervertebral disc is involved in distributing mechanical forces along the spinal column while imparting flexibility. This feature arises from the hydrostatic pressure established within a sealed nucleus pulposus. The nucleus pulposus (NP) is composed mainly of proteoglycans and type II collagen fibers that are gently arranged and absorbs a relatively large amount of water. The nucleus pulposus is laterally surrounded by a fibrocartilage layer called annulus fibrosus (AF). Finally, the intervertebral disc is covered by a thin layer of hyaline cartilage on the boundary with each vertebra, that is, an endplate.

It is suggested that native nucleus pulposus cells change their phenotype from vacuolated notochord cells to active nucleus pulposus cells, senescent cells, and fibrous nucleus pulposus cells, to form a lot of heterogeneous cell populations in the nucleus pulposus. Such changes to different cell types seem to play a decisive role in the progression of intervertebral disc degeneration. Due to aging and biological and mechanical stress (wear), the nucleus pulposus shows a gradual decrease in Tie2/GD2-positive progenitor cells (Non Patent Literature 1: Sakai et al., 2012), and the cells are aged to be switched to fibrous cells. These changes result in a change in a proteoglycan-rich nucleus pulposus extracellular matrix into a fibrous structure, thereby deteriorating the hydrostatic pressure and other biomechanical characteristics of the intervertebral disc. A cascade of these events potentially causes low back pain and other spinal disorders. Conversely, intervertebral disc degeneration and related disorders are rarely seen in other animal models (such as pigs, mice, and rats). This is likely due to the facts that these animals maintain the original notochordal cell phenotype, and as such are able to maintain healthy intervertebral discs with an active phenotype of nucleus pulposus cells, which play a decisive role in preventing the intervertebral disc disorders. Accordingly, for treating human intervertebral disc degeneration or the like or alleviating pains and disorders, a strong research emphasis is placed on establishing strategies to repopulate or induce proliferation of endemic cell populations in the intervertebral disc tissue to actively produce and regenerative intervertebral disc matrix.

As part of such strategies, various studies show the advantage of transplanting active nucleus pulposus cells or chondrocytes into affected areas by intervertebral disc degeneration or the like (as reviewed in, Non Patent Literature 2: Schol and Sakai, 2018), and it is regarded as a promising option for treatment of intervertebral disc degeneration or the like. Nevertheless, supply of active nucleus pulposus cells or the like for cell transplantation is insufficient both clinically and scientifically. Conventionally, the possibility of regenerating intervertebral discs by transplanting autologous or allogeneic donor nucleus pulposus cells that are isolated from an intervertebral disc surgically removed during surgery or chondrocytes that essentially have the ability to produce an extracellular matrix like the nucleus pulposus cells in the intervertebral disc environment has been explored. However, since the intervertebral discs or cartilage collected from donors by surgery or the like are often damaged due to diseases, trauma. aging, or the like, there is a possibility that nucleus pulposus cells or chondrocytes contained therein do not have sufficient effectiveness as transplantation regeneration materials (Non Patent Literature 1). Further, in the treatment method of transplanting nucleus pulposus cells, donor cells are reactivated and generated by in-vitro culture to prepare the final purified product of active nucleus pulposus cell populations, but the traits of nucleus pulposus cells are known to be lost (dedifferentiated) by culture, and it is a technical problem to amplify nucleus pulposus cells without dedifferentiation (Non Patent Literature 1).

As cells to be transplanted into the intervertebral disc, it is also conceivable to use stem cells capable of differentiating into nucleus pulposus cells, such as mesenchymal stem cells (MSCs) that are derived from bone marrow, placenta, fat. or the like, comparatively easily accessible, and available in a large amount. However, the hypoglycemia, hyperosmolarity, and hypoxic environment caused by avascular and progressive degeneration of an intervertebral disc is a significant obstacle to the survival and thriving of MSCs. Therefore, it is unknown to what extent the transplanted MSCs can actively produce and secrete matrix proteins or cytokines for reactivating surrounding cells to regenerate the intervertebral disc tissue for long-term effects, and thus clinical application of MSCs to intervertebral disc disorders is limited (Non Patent Literature 3: Fang, Z., et al., 2013). There are similar problems in pluripotent stem cells other than mesenchymal stem cells, such as hematopoietic stem cells.

Therefore, a technique for artificially maintaining or inducing the traits (phenotype) of active nucleus pulposus cells is required, but information on transcription factors and their control, which is the key of the technique, is limited, and induction of the active nucleus pulposus cell phenotype itself in vitro has not been realized so far. Conventionally, the possibility of obtaining a nucleus pulposus cell-like phenotype as an alternative to actual nucleus pulposus cells by applying various stimuli to cells that seem promising among cells other than nucleus pulposus cells has been studied. As such candidate cells, induced pluripotent stem cells (iPS cells) are used other than MSCs, although less frequently. It is known that various cells can be obtained from such pluripotent or multipotent stem cells by differentiation induction under appropriate conditions. In order to obtain a nucleus pulposus cell-like phenotype from pluripotent or multipotent stem cells, stimulation by extracellular matrix compositions, oxygen partial pressure, mechanical or osmotic pressure, application of scaffold such as hydrogel, stimulation by growth factors or cytokines, stimulation by co-culture with other cells or culture supernatant, or combinations thereof are attempted. These studies have generally reported that the expression level of nucleus pulposus cell-related genes such as type II collagen (COL2), aggrecan (ACAN), and differentiation cluster 24 (CD24) can be enhanced, or the production of those proteins can be improved. Further, it has been also reported that cell encapsulation, aggregation, or direct transplantation in vivo causes deposition of an extracellular matrix similar to an NP-cell like extra-cellular matrix rich in proteoglycans and COL2, which is generally reported as regeneration of an intervertebral disc.

In general, there is a concern about the reproducibility in the method using growth factors. In order to elicit a desired response by applying growth factors, cells need to present the correct cell membrane-bound receptors that correspond to the growth factors. The presentation of appropriate receptors is highly specific to the cell type and donor, thus potentially causing problems in reproducibility of the induction procedure. Further, it is still unknown whether there is a possibility that cell transplantation in an environment with no or different growth factors (that is, in an environment in the original nucleus pulposus tissue or the like), can negatively alter the transplanted cell phenotype. Finally, continuous supplementation of growth factors accounts for a relatively expensive part of a culture process for producing or using induced nucleus pulposus cells, thereby further limiting the clinical applicability.

Only a few studies have explored the possibility of fabricating an active nucleus pulposus cell phenotype from pluripotent or multipotent cells, such as MSCs and iPSCs, or terminally differentiated cells other than nucleus pulposus cells by more direct manipulation to the gene expression profile, instead of adjusting the culture conditions as above, and information on transcription factors and their control, which is the key of such gene operation. has been exceptionally limited.

For example. Non Patent Literature 4 (Xu et al., 2016) discloses that bone morphogenetic protein 7 (BMP7) was overexpressed in rabbit bone marrow-derived MSCs, in order to stimulate differentiation into NP cells by enhancing secretion of BMP7, as a result of which, the expressions of COL2, ACAN, SOX9, keratin (KRT)-8, and KRT19 were enhanced at the mRNA level by monolayer culture two to three weeks after transfection. However, when a cell product obtained in this method was transplanted into a rabbit model with intervertebral disc degeneration, a beneficial effect was observed in both the sham control and the BMP7 transfection group 6 weeks and 12 weeks after transplantation, but the glycosaminoglycan/DNA ratio in the BMP7overexpression group at the 12th week were merely slightly increased as compared with the sham control.

Non Patent Literature 5 (Chen et al., 2017) discloses that, by the same approach, an established factor WNT11 was overexpressed by lentivirus transduction in rat fat-derived MSCs, and an in-vitro evaluation thereof showed a slight but significant increase in COL2, ACAN, and SOX9 at both the mRNA and protein levels, as compared with the sham control was transfected to overexpress green fluorescent protein (GFP).

As another approach, Non Patent Literature 6 (Outani et al., 2013) discloses that introduction of SOX9, known as a master regulator transcription factor for forming cartilage into fibroblasts (Non Patent Literature 7: Wright et al., 1995, and Non Patent Literature 8: Bi et al., 1999), enabled direct reprogramming of their cells into a chondrocyte phenotype.

Non Patent Literature 9 (Yang et al, 2011) discloses that a cell line overexpressing SOX9 was established in rat adipose tissue-derived MSCs by a leukemia virus-derived viral vector pseudotyped with vesicular stomatitis virus G-envelope glycoprotein. Further, it also discloses that, as a result of culturing the established cell line with or without addition of transforming growth factor β (TGFβ)-3, the ratios GAG/DNA and COL2/DNA changed, and such a result showed that the overexpression of SOX9 could induce a relative increase in the ratios of GAG and COL2 with supplementation of TGFβ3, conversely. manipulation by only any one of supplementation of TGFβ3 to the culture medium or transduction with SOX9 would not result in a significant improvement. However, the literature does not present any further evaluation regarding a nucleus pulposus cell phenotype.

Likewise, Non Patent Literature 10 (Sun et al. 2014) also discloses that rabbit bone marrow-derived MSCs were transduced with SOX9 through an adenoviral vector. In an in-vitro monolayer evaluation, the transduced cells clearly showed an obviously strong increase in expressions of ACAN, COL2, and SOX9 at the mRNA level but showed only a slight decrease in type 1 collagen (COL1), as compared with the GFP-control group. The same results were obtained from transduced cells cultured using chitosan-glycerophosphate gel. Finally, when transduced cells were transplanted into a rabbit model with induced intervertebral disc degeneration, both a GFP-expressing MSC-administered group and a SOX9-overexpressing MSC-administered group showed improved T2 intensity, which showed the advantage of MSC transplantation again, but the SOX9-overexpressing MSC-administered group showed a small but significant improvement as compared with the GFP-expressing cells. Likewise, the histological results also revealed a benefit by MSC transplantation with a slight increase due to the SOX9 overexpression, as shown by safranin-O) staining (representing the content of proteoglycan), and COL2 targeted staining, in contrast to the GFP expression.

Non Patent Literature 11 (Hiramatsu et al., 2011) discloses that mouse adult dermal fibroblasts were differentiated using combinations of reprogramming factor c-MYC with transcription factors KLF4 and SOX9, and as a result of overexpression of their genes, expressions of ACAN and COL2 specific to chondrocytes could be stimulated, despite the mature differentiated state of starting cell populations.

Non Patent Literature 12 (Outani et al, 2013, by the same group as Non Patent Literature 11) also discloses evidence of successful reprogramming from fibroblasts to a chondrocyte phenotype by overexpression of KLF4, c-MYC and SOX9. This literature discloses that expressions of ACAN and COL2 were enhanced by application to dermal fibroblasts derived from humans. Further, it is also revealed that transplantation of the reprogrammed fibroblasts enhanced deposition of a chondrogenic matrix in a mouse model with any one of subcutaneous and chondrogenic defect indications.

The aforementioned conventional art literatures generally indicate that expression of transcription factor SOX9 can stimulate the chondrogenic phenotype. Accordingly, overexpression of SOX9 highly possibly results in a more common chondrocyte phenotype rather than a specific nucleus pulposus cell phenotype. The conventional art literatures did not examine markers or morphological characteristics specifically associated with nucleus pulposus cells, as a comparison with other cells exhibiting cartilage traits.

In addition, as markers associated with the traits of nucleus pulposus cells, hypoxia-inducible factor 1 sub-unit α (HIF1α) (Non Patent Literature 13: Risbud et al., 2006), Brachyury (T) (Non Patent Literature 14: Sheyn et al., 2017), Paired Box 1 (PAX1) (Non Patent Literature 15: Risbud et al., 2015), and the like have been reported, but their gene expression profiles have not been sufficiently evaluated so far.

### CITATION LIST

### Non Patent Literature

[Non Patent Literature 1] Sakai, D. et al. Exhaustion of nucleus pulposus progenitor cells with ageing and degeneration of the intervertebral disc. Nature communications 3, 1264, doi: 10.1038/ncomms 2226 (2012).
[Non Patent Literature 2] Schol J, Sakai D. Cell therapy for intervertebral disc herniation and degenerative disc disease: clinical trials. International orthopaedics. (2018) doi: https://doi.org/10.1007/s00264-018-4223-1.
[Non Patent Literature 3] Fang, Z., et al., Differentiation of GFP-Bcl-2-engineered mesenchymal stem cells towards a nucleus pulposus-like phenotype under hypoxia in vitro. Biochem Biophys Res Commun, 2013. 432 (3): p. 444-50.
[Non Patent Literature 4] Xu, J., et al., BMP7 enhances the effect of BMSCs on extracellular matrix remodeling in a rabbit model of intervertebral disc degeneration. FEBS J. 2016. 283 (9): p. 1689-700.
[Non Patent Literature 5] Chen, H.T., et al., Wnt11 overexpression promote adipose-derived stem cells differentiating to the nucleus pulposus-like phenotype. Eur Rev Med Pharmacol Sci, 2017. 21 (7): p. 1462-1470.
[Non Patent Literature 6] Outani, H. et al. Direct induction of chondrogenic cells from human dermal fibroblasts cultured by defined factors (PloS one, 2013).
[Non Patent Literature 7] Wright. E., et al., The Sry-related gene Sox9 is expressed during chondrogenesis in mouse embryos. Nat Genet, 1995. 9 (1): p, 15-20.
[Non Patent Literature 8] Bi, W., et al., Sox9 is required for cartilage formation. Nat Genet, 1999. 22 (1): p. 85-9.
[Non Patent Literature 9] Yang. Z., et al., Sox-9 facilitates differentiation of adipose tissue-derived stem cells into a chondrocyte-like phenotype in vitro. J Orthop Res, 2011. 29 (8): p. 1291-7.
[Non Patent Literature 10] Sun, W., et al., Sox9 gene transfer enhanced regenerative effect of bone marrow mesenchymal stem cells on the degenerated intervertebral disc in a rabbit model. PLOS One, 2014. 9 (4): p. e93570.
[Non Patent Literature 11] Hiramatsu, K., et al., Generation of hyaline cartilaginous tissue from mouse adult dermal fibroblast culture by defined factors. J Clin Invest, 2011. 121 (2): p, 640-57.
[Non Patent Literature 12] Outani, H., et al., Direct induction of chondrogenic cells from human dermal fibroblast culture by defined factors. PLOS One, 2013. 8 (10): p. e 77365.
[Non Patent Literature 13] Risbud, M.V., et al., Nucleus pulposus cells express HIF-1 alpha under normoxic culture conditions: a metabolic adaptation to the intervertebral disc microenvironment. JC ell Biochem, 2006.98 (1): p. 152-9.
[Non Patent Literature 14] Sheyn et al (2017) Human iPS-derived notochordal cells survive and retain their phenotype in degenerated porcine IVD-ORS 2017 Annual Meeting Paper No.0051.
[Non Patent Literature 15] Risbud, M.V., et al., Defining the phenotype of young healthy nucleus pulposus cells: recommendations of the Spine Research Interest Group at the 2014 annual ORS meeting. J Orthop Res, 2015. 33 (3): p. 283-93.

### SUMMARY OF INVENTION

### Technical Problem

As described above, there are various problems in transplanting intervertebral disc (nucleus pulposus) cells or chondrocytes collected from donors, and also in transplanting stem cells capable of differentiating into nucleus pulposus cells such as mesenchymal stem cells and hematopoietic stem cells, in treating intervertebral disc degeneration or the like. Further, there are also problems of reproducibility and cost in the method of differentiation induction of stem cells such as MSCs into an active nucleus pulposus cell phenotype using specific growth factors, and there is no evidence for achieving the safety or therapeutic effects that counteract such problems.

Therefore, it seems ideal to perform differentiation induction into an active nucleus pulposus cell phenotype by allowing specific transcription factors to act on stem cells or the like (that is, by changing transcription factor profiles), and to use the active nucleus pulposus cell phenotype thus obtained for transplantation. However, “nucleus pulposus cell master regulator transcription factors” that enable such differentiation induction, keep exerting sufficient functions as an active nucleus pulposus cell phenotype also after transplantation of the active nucleus pulposus cell phenotype obtained (such as producing a sufficient amount of extracellular matrix), and thus can be clinically effective have not been established so far. Further, potent transcription factors (master regulator transcription factors) that enable an active nucleus pulposus cell phenotype to be obtained by transdifferentiation from terminally differentiated cells other than nucleus pulposus cells, not from stem cells such as MSCs, have still not been found. SOX9, which has been established as a master regulator transcription factor for cartilage formation, has not been demonstrated to have sufficient usefulness as a master regulator transcription factor for an active nucleus pulposus cell phenotype.

It is an object of the present invention to provide reproducible means that enables the production of an active nucleus pulposus cell phenotype from desired cells such as terminally differentiated cells or pluripotent or multipotent stem cells.

### Solution to Problem

The inventors screened transcription factors specific to nucleus pulposus cells by microarray assay, iPS cell interference assay, siRNA assay, or the like as described in Examples below, instead of using the transcription factor SOX9 (chondrogenic master regulator) associated with cartilage formation as a starting point as in conventional arts. Further, the inventors have found that a combination of specific transcription factors selected from the candidates, typically, a combination of at least two selected from the group consisting of Brachyury (T), SRY-box6 (SOX6), and Forkhead Box Q1 (FOXQ1) serve as a set of potent transcription factors which should be called “nucleus pulposus cell master regulator transcription factors” that enable induction into an active nucleus pulposus cell phenotype not only by differentiation induction from stem cells such as MSCs but also transdifferentiation, for example, from terminally differentiated cells such as fibroblasts, thereby accomplishing the present invention.

That is, the present invention provides at least the following items.
(1) A differentiation inducer that is an agent comprising an effective amount of a gene of transcription factors for differentiation induction of nucleated cells other than active nucleus pulposus cell phenotypes into an active nucleus pulposus cell phenotype (hereinafter referred to as “nucleus pulposus cell master regulator transcription factors”) or a product thereof (hereinafter referred to as “differentiation inducer”), wherein the master regulator transcription factors comprise at least two selected from the group consisting of Brachyury (T), SRY-box6 (SOX6), and Forkhead Box Q1 (FOXQ1), or homologs thereof.
(2) The differentiation inducer according to item (1), wherein the nucleus pulposus cell master regulator transcription factors further comprise at least one selected from the group consisting of Paired Like Homeodomain1 (PITX1) and Paired Box 1 (PAX1), or a homolog thereof.
(3) The differentiation inducer according to item (1) or (2), wherein the nucleus pulposus cell master regulator transcription factors further comprise at least one selected from the group consisting of Hypoxia inducible factor 3 alpha (HIF3a), SRY-box9 (SOX9), Runt-related Transcription Factor 1 (RUNX1), hypoxia Inducible Factor 1 alpha (HIF1α), and Forehead Box A2 (FOXA2), or a homolog thereof.
(4) The differentiation inducer according to any one of items (1) to (3), wherein the nucleus pulposus cell master regulator transcription factors are in the form of a gene inserted into an expression vector.
(5) A pharmaceutical composition for treating or preventing an intervertebral disc disorder in a vertebrate animal, comprising the differentiation inducer according to any one of items (1) to (4).
(6) A method for producing induced nucleus pulposus cells, comprising the steps of: introducing the differentiation inducer according to any one of items (1) to (4) in vitro into nucleated cells other than active nucleus pulposus cell phenotypes (hereinafter referred to as “introduction step”); and performing transdifferentiation or differentiation induction into an active nucleus pulposus cell phenotype through culturing the transcription factor introduction cells obtained by the introduction step (hereinafter referred to as “differentiation induction step”).
(7) The method for producing induced nucleus pulposus cells according to item (6), further comprising a step of checking an expression status of at least one selected from the group consisting of CD24, aggrecan, and type II collagen in the cells during culture or after culture in the differentiation induction step.
(8) The method for producing induced nucleus pulposus cells according to item (7), wherein the differentiation induction step comprises culturing the transcription factor introduction cells in a culture medium supplemented with transforming growth factor β1 (TGFβ1) and growth differentiation factor 5 (GDF5).
(9) The method for producing induced nucleus pulposus cells according to item (7) or (8), wherein the differentiation induction step comprises culturing the transcription factor introduction cells under at least one condition selected from the group consisting of a hypoxic environment, an acidic environment, and a low-glucose environment.
(10) Transcription factor introduction cells that are cells comprising an effective amount of the nucleus pulposus cell master regulator transcription factors defined in any one of items (1) to (4).
(11) Induced nucleus pulposus cells that are cells having an active nucleus pulposus cell phenotype obtained through culturing the transcription factor introduction cells according to item (10).
(12) The induced nucleus pulposus cells according to item (11), wherein the induced nucleus pulposus cells are expressing at least one selected from the group consisting of CD24, aggrecan, and type II collagen.
(13) The induced nucleus pulposus cells according to item (11) or (12), wherein the induced nucleus pulposus cells are viable under at least one condition selected from the group consisting of a hypoxic environment, an acidic environment, and a low-glucose environment.
(14) The induced nucleus pulposus cells according to any one of items (11) to (13). wherein the induced nucleus pulposus cells have intercellular vacuoles.
(15) A cell population comprising the transcription factor introduction cells according to item (10) and/or the induced nucleus pulposus cells according to any one of items (11) to (14).
(16) A cell preparation for treating or preventing an intervertebral disc disorder in a vertebrate animal, comprising the induced nucleus pulposus cells according to any one of items (11) to (14) or the cell population according to item (15).
(17) A method for treating or preventing an intervertebral disc disorder in a vertebrate animal (excluding human), comprising transplanting or administering the induced nucleus pulposus cells according to any one of items (11) to (14), the cell population according to item (15), or the cell preparation according to item (16) in vivo so as to act on the intervertebral disc nucleus pulposus tissue.
(18) A method for treating or preventing an intervertebral disc disorder in a vertebrate animal (excluding human), comprising administering the differentiation inducer according to any one of items (1) to (4) or the pharmaceutical composition according to item (5) in vivo so as to act on nucleus pulposus cells in an intervertebral disc.
(19) A method for screening for a medicine or a method for treating or preventing an intervertebral disc disorder in a vertebrate animal, comprising a step of testing effectiveness and safety in a subject using the transcription factor introduction cells according to item (10), the induced nucleus pulposus cells according to any one of items (11) to (14), or the cell population according to item (15).
(20) A method for obtaining an indicator associated with an aging, degenerating or disease state of nucleus pulposus cells, comprising measuring expression levels of the nucleus pulposus cell master regulator transcription factors defined in any one of items (1) to (4) in isolated nucleus pulposus cells.

In the aforementioned items, the provision that the application target of the invention “excludes humans” is made only in view of industrial applicability, and the target can “include human” from a technical point of view.

### Advantageous Effects of Invention

The present invention enables cell transcription profiles to be directly changed, that is, cell transcription factor profiles to be induced into the same profiles as those of nucleus pulposus cells by specifically stimulating the master regulator of an active nucleus pulposus cell phenotype using specific transcription factors. This approach enables cells to be directly modified without the need for specific cell membrane receptors or related signaling proteins and therefore can be implemented relatively in-expensively with reliability (reproducibility). Further, even disseminated vacuolar presenting cells have a morphology similar to that of nucleus pulposus cells.

Further, the nucleus pulposus cell master regulator transcription factors specified by the present invention are potent and thus expand the range of cells to which direct reprogramming can be applied. In the conventional art, pluripotent or multipotent cell types (such as MSCs) were necessary, but the present invention also enables transdifferentiation of mature differentiated cells such as human dermal fibroblasts that have been terminally differentiated. A clear up-regulation can be given to extracellular matrix components such as type II collagen and aggrecan only by using the nucleus pulposus cell master regulator transcription factors specified by the present invention without using SOX9 that is a master regulator for cartilage formation. Accordingly, induced nucleus pulposus cells to be obtained by the present invention can survive under a severe nucleus pulposus microenvironment and can actively produce a nucleus pulposus-related extracellular matrix such as proteoglycans and type II collagen. The present invention can be further implemented by an embodiment of introducing the nucleus pulposus cell master regulator transcription factors into senescent cells or fibrous nucleus pulposus cells contained in an intervertebral disc or dedifferentiated nucleus pulposus cells during culture, to “regenerate” the cells into an active nucleus pulposus cell phenotype.

Currently, effective treatment for chronic low back pain or the like is limited to symptomatic therapy, but application of the present invention enables mass supply of superior cell products for intervertebral disc regenerative medicine, thereby solving the problem of the number and quality of donors and the problem of production cost. The situation in which nucleus pulposus cells to be transplanted are collected from a tissue removed from affected areas of intervertebral disc degeneration such as scoliosis and intervertebral disc hernia or other organs, as before, can be overcome, and functional nucleus pulposus cells that are more healthy and closer to the original traits can be fabricated from various somatic cell types such as cells contained in the skin or adipose tissue. Further, nucleus pulposus cells to be fabricated by the present invention can also be autologous, and therefore the risk of using non-autologous cells can be avoided.

According to another aspect, induced nucleus pulposus cells to be obtained by the present invention can be fabricated from healthy individual donor cells and therefore can also be used for studying the health state of nucleus pulposus of young individuals under in-vitro conditions. Further, the present invention enables nucleus pulposus cells derived from patients presenting specific gene mutations that affect the spinal or general cell biology to be fabricated. That is, the present invention enables induced nucleus pulposus cells to be fabricated from cells contained in easy-to-access tissue sources such as the skin or adipose tissue of patients, and enables how gene mutations in the original cells affect the behavior of nucleus pulposus cells in vitro to be evaluated. Finally, use of induced nucleus pulposus cells to be obtained by the present invention in a personalized medical care strategy enables how medicine that becomes a treatment candidate and its dose affect induced nucleus pulposus cell populations specific to the patient to be determined, thereby revealing patient-specific potential negative effects before actual administration to the patient, which can be useful for knowing an appropriate treatment method.

### BRIEF DESCRIPTION OF DRAWINGS

FIG. 1 is a conceptual diagram showing the gist and application of the present invention based on examples and typical embodiments described in this description. Transcription factors that are important for maintaining or inducing a phenotype from human nucleus pulposus cells were examined through various screening and reprogramming assays. In the process, promising sets and combinations of transcription factors are specified. For example, the combination of T, SOX6, and FOXQ1 can strongly induce the properties associated with nucleus pulposus cells. These transcription factors can be applied to a method for transdifferentiation, that is, intended reprogramming, and can change mesenchymal stem cells (MSCs), fibroblasts, intermediate nucleus pulposus cells, or the like into an active nucleus pulposus cell phenotype in vitro or in situ. Further, the induced nucleus pulposus cells obtained can be used as a cell population for studies or cell transplantation in strategies to recover an intervertebral disc that has degenerated, for example.

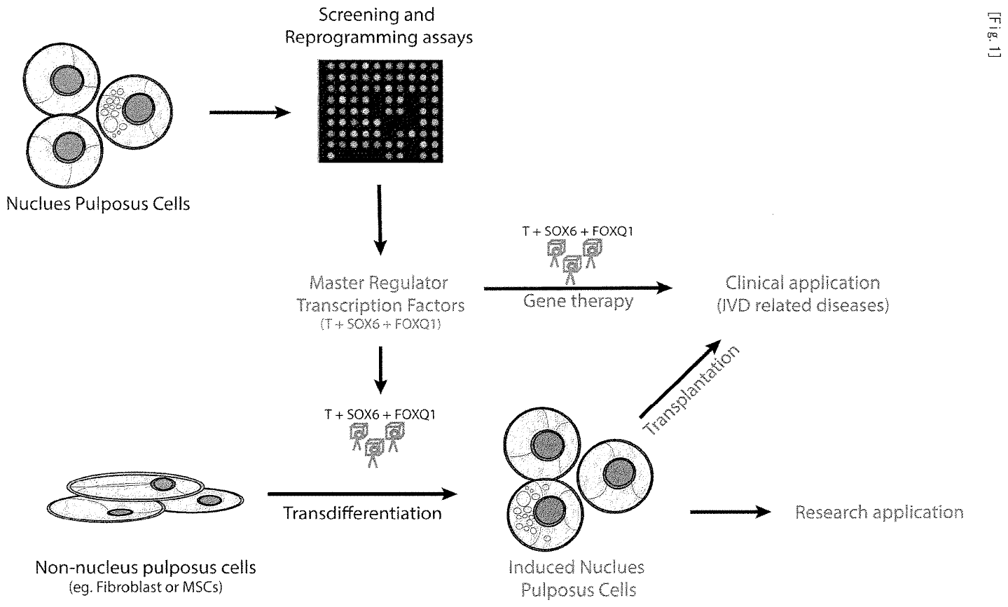

FIG. 2 is a heat map showing the overview of expression profiles of relative transcription factors in a fractionated human nucleus pulposus cell population, which have been evaluated by comparative microarray analysis. The mRNA expression level of each transcription factor was analyzed for CD24, GD2, and TIE2-positive nucleus pulposus cells (which are respectively 2D CD24, 2D GD2, and 2D TIE2 in the figure) obtained by monolayer culture and CD24-positive cells (3D CD24 in the figure) derived from a methylcellulose spheroid colony-forming cell unit and obtained by 3D pellet culture. Expression values are individually presented as a difference in mRNA expression level of each transcription factor, as compared with the level in human dermal fibroblasts (Fibro, the leftmost column in the figure), human neural progenitor cells (NPC, the 2nd column from the left in the figure), human lung cells (Lung, the 3rd column from the left in the figure), and human induced pluripotent stem cells (iPSCs, the rightmost column in the figure). Positive values indicate higher expression levels in the nucleus pulposus cells, and negative values indicate lower expression levels in the nucleus pulposus cells.

FIG. 3 is a plot showing the overview of iPS cell interference assays representing the evaluation of effectiveness of transcription factors that are ordered based on the interference with colony formation in induced pluripotent stem cells (iPSCs). The nucleus pulposus cells were transduced with four Yamanaka factors established and further transcription factors. Supposing that the score of the number of colonies formed in nucleus pulposus cell cultures transduced only with the four Yamanaka factors was 1.000 (reference value), the score of the number of colonies formed in nucleus pulposus cell cultures transduced with the four Yamanaka factors combined with each transcription factor was presented as a relative value to the reference value. It is indicated that transcription factors having a score below 1.000 have an effect of blocking induction into iPS cells, and that transcription factors having a score over 1.000 have an effect of promoting induction into iPS cells. The images of stained iPS cell colonies on the right side of the plot are typical examples representing relative colony formation efficiencies.

FIG. 4 is a heat map showing the results of examining the influence of down-regulation due to the master regulator transcription factor candidates based on the measurement of the relative mRNA expression levels of nucleus pulposus cell markers in human nucleus pulposus cells. When RNA interference mediated by siRNA was performed on transcription of the master regulator transcription factor candidates, the expression levels of the nucleus pulposus cell markers changed. All values were calculated as relative values to the GAPDH expression level and then were compared with the respective SHAM controls (2^{ΔΔCT).}

FIG. 5 includes optical micrographs (magnification 10×, each scale bar represents 250 μm) of induced nucleus pulposus cells obtained by transducing (A) fibroblasts and (B) MSCs with the master regulator transcription factors (four combinations of T, SOX6, and FOXQ1), followed by one-week culture. (A) Induced nucleus pulposus cells fabricated from human fibroblasts were contrasted with the respective SHAM controls (which were transduced with green fluorescent protein (GFP) instead of the master regulator transcription factors). (B) Likewise, induced nucleus pulposus cells fabricated from human bone marrow-derived MSCs were contrasted with the SHAM control.

FIG. 6 includes optical micrographs (magnification 10× and 20×) of vacuoles observed within induced nucleus pulposus cells (in the protoplasm) one to two weeks after transduction. Human newborn dermal fibroblasts were transduced with three combinations of T, SOX6, and FOXQ1, and the SHAM control was transduced with GFP, instead. The black arrows point to the vacuoles within the cells.

FIG. 7 shows the results of examining the mRNA expression levels of nucleus pulposus cell markers (aggrecan (ACAN), type II collagen (COL2), type I collagen (COL1), CD24, keratin 18 (KRT18), and keratin 8 (KRT8)) by different combinations of master regulator transcription factors in induced nucleus pulposus cells derived from fibroblasts. In each graph, human dermal fibroblasts derived from three different donors were transduced with different combinations of T, SOX6 (S), and FOXQ1 (F), cultured for one week, and contrasted with the respective SHAM controls transduced with GFP. The mRNA expression level was calculated as a relative value to the GAPDH expression level, and a difference from the expression level of the SHAM control (relative value) was determined for each donor. The numerical value represents an average±standard error (SEM). For the statistical analysis, two-way analysis of variance (no matching) and Tukey's multiple comparison test were used, and p<0.05 was determined to be a significant difference (*p≤0.05, **p≤0.01, ***p≤0.005, ****p≤0.001).

FIG. 8 includes optical micrographs (each scale bar represents 250 μm) showing the histological overview of induced nucleus pulposus cells in chondrogenic pellets. Hematoxylin/eosin and safranin-O/fast green stained sections of pellet cultures of fibroblasts or MSCs transduced with different combinations of brachyury (T), SOX6 (S), PITX1 (P), and FOXQ1 (F) were contrasted with the respective SHAM controls transduced with GFP. Fibroblasts transduced with a combination of TS and TSP, and MSCs transduced with TS show strong cell vacuolation by hematoxylin/eosin staining. Meanwhile, the combination of TS and TSF in induced nucleus pulposus cells derived from both the fibroblasts and the MSCs showed a proteoglycan region or uniform deposition by red safranin-O staining.

FIG. 9 shows the results of examining the mRNA expression levels of nucleus pulposus cell markers (aggrecan (ACAN), type II collagen (COL2), keratin 8 (KRT8), and CD24) by different combinations of master regulator transcription factors in induced nucleus pulposus cells derived from MSCs. The MSCs were transduced with different combinations of brachyury (T), SOX6 (S), and FOXQ1 (F), and each SHAM control was transduced with GFP. The cells after transduction were differentiated by culture for two weeks. Each mRNA expression level was calculated as a relative value to the GAPDH expression level and then was calculated as a relative value to the expression level of the SHAM control cultured in a culture medium with no growth factors added. For the statistical analysis, two-way analysis of variance (no matching) and Tukey's multiple comparison test were used, and p<0.05 was determined to be a significant difference (*p≤0.05, **p≤0.01, **p≤0.005, ****p≤0.001).

FIG. 10 (FIG. 10-1, FIG. 10-2, and FIG. 10-3) includes fluorescence micrographs (magnification 10× and 40×, the scale bars respectively represent 200 μm and 50 μm) in the immunohistochemistry analysis of pellet cultures of induced nucleus pulposus cells derived from MSCs. (A) Fluorescence stained images of aggrecan (ACAN). (B) Fluorescence stained images of type II collagen (COL2). The induced nucleus pulposus cells were transduced with a combination (TSF) of brachyury (T) and SOX6 (S) supplemented with FOXQ1 (F), and a combination (TS) without supplementation, and each SHAM control was transduced with GFP. Staining revealed that all the nucleus pulposus cell markers examined were specifically stained in the case of transduction with all the master regulator transcription factors. The SHAM control within a pellet merely showed sporadic staining of the nucleus pulposus cell markers with no or low intensity. In contrast, the MSCs transduced with TS or TSF showed staining of all the nucleus pulposus cell markers with comparatively high intensity throughout a pellet. The reason why the MSCs transduced with TS or TSF express GFP is because GFP is contained in a T vector as a reporter gene.

FIG. 10-2 Same as above. (C) Fluorescence stained images of keratin 18 (KRT18). (D) Fluorescence stained images of PITX1.

FIG. 10-3 Same as above. (E) Fluorescence stained images of PAX1. (F) Fluorescence stained images of CD24.

FIG. 11 includes graphs showing the relative mRNA expression levels (calculated by 2^{-ΔΔCT}) of nucleus pulposus cell markers (aggrecan (ACAN), type II collagen (COL2), and CD24) when contrasted with SHAM controls using GAPDH as the control gene. The expression levels were measured after transducing fibroblasts with T and another transcription factor (PAX1, RUNX1, HIF3α, PITX1, FOXA1, HIF1α, SOX6, FOXQ1, and SOX9) and culturing the fibroblasts for two weeks in a culture medium supplemented with GDF5 at 100 ng/ml and TGFβ1 at 10 ng/ml. As compared with the cases of the SHAM controls transduced so as to express GFP and the cases of transduction with T alone, the expression levels of fibroblasts transduced with the pairs of transcription factors increased. The bar indicates the average of cells in each sample (n=1).

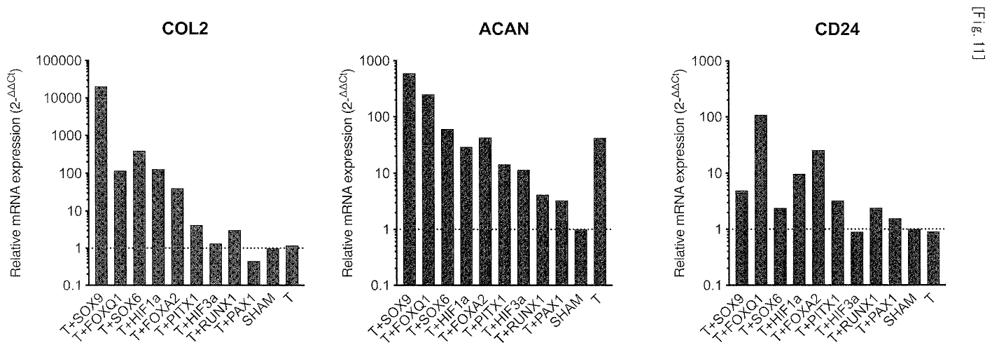

FIG. 12 includes graphs and images representing the evaluation results for the optimal combination of growth factors for inducing a nucleus pulposus cell phenotype. (A) shows relative values of the gene expression levels of ACAN, COL2A1, CA12, CD24, and Vimentin two weeks after monolayer culture of MSCs, when a combination of GDF5 or GDF6 with TGFβ1, TGFβ2, or TGFβ3 was added. Each expression level of ACAN, COL2A1, CA12, CD24, and Vimentin is a correction value with reference to the expression level of housekeeping gene GAPDH and is shown as a relative value with reference to a correction value (1.0) with no growth factors added (NC). n=1. (B) shows each MSC pellet with a combination of GDF5 or GDF6 supplemented with TGFβ1, TGFβ2, or TGFβ3, stained with Safranin-O and Fast green three weeks after culture. Each scale bar represents 500 μm.

### DESCRIPTION OF EMBODIMENTS

### Differentiation Inducer (Nucleus Pulposus Cell Master Regulator Transcription Factors)

The differentiation inducer of the present invention is an agent containing a gene of transcription factors (nucleus pulposus cell master regulator transcription factors) or a product thereof in an effective amount for differentiating cells into an active nucleus pulposus cell phenotype. In the present invention, “inducing cells into an active nucleus pulposus cell phenotype” includes embodiments such as (i) maintaining “trans-differentiation” into nucleus pulposus cells from terminally differentiated cells other than nucleus pulposus cells or cells committed to differentiation into cells other than nucleus pulposus cells and inducing the cells into an active nucleus pulposus cell phenotype (which may be referred to as “first embodiment” in this description), (ii) differentiation induction into an active nucleus pulposus cell phenotype from stem cells or the like capable of differentiating into nucleus pulposus cells and other cells (having pluripotency or multipotency) (which may be referred to as “second embodiment” in this description), and (iii) reactivating dedifferentiated or otherwise compromised nucleus pulposus cells into an active nucleus pulposus cell phenotype (which may be referred to as “third embodiment” in this description). The differentiation inducer of the present invention can be used in any form of the first, second, and third embodiments, and the term “differentiation inducer” is used as a general term, but it is also possible to use the term “transdifferentiation agent” particularly for use in the first embodiment, the term “differentiation inducer” particularly for use in the second embodiment, and the term “reactivation agent” particularly for use in the third embodiment.

Examples of the nucleus pulposus cell master regulator transcription factors as the subject of the present invention, that is, the master regulator transcription factors that are important for maintaining an active nucleus pulposus cell phenotype include Brachyury (T), SRY-box 6 (SOX6), Forkhead Box Q1 (FOXQ1), Paired Like Homeodomain 1 (PITX1), Paired Box Protein1 (PAX1), Hypoxia inducible factor 3 alpha (HIF3α), SRY-box9 (SOX9), Krueppel-like factor 6 (KLF6), Runt-related Transcription Factor 1 (RUNX1), hypoxia Inducible Factor 1 alpha (HIF1α), and Forehead Box A2 (FOXA2), and homologs thereof (which may be referred to as “transcription factor group of the present invention” in this description). The homolog of each master regulator transcription factor is known to those skilled in the art and can be searched by databases such as Japan DNA data bank (DDBJ), NCBI BenBank, and EMBL. In the present invention, any one of these nucleus pulposus cell master regulator transcription factors may be used alone, or two or more, three or more, four or more, or more types of them may be optionally combined for use. Which (combination of) nucleus pulposus cell master regulator transcription factors are to be selected can be appropriately adjusted corresponding to whether the differentiation inducer of the present invention is to be used as a transdifferentiation agent (for terminally differentiated cells or the like other than nucleus pulposus cells) or as a differentiation inducer (for stem cells or the like capable of differentiating into nucleus pulposus cells and other cells) or as a reactivation agent (for dedifferentiated or damaged nucleus pulposus cells).

In the present invention, the “active nucleus pulposus cell phenotype” means to have one or more traits selected from (i) producing a large amount of nucleus pulposus-related matrix proteins such as proteoglycans (for example, aggrecan) and type II collagen or expressing at least one of cell markers specific to nucleus pulposus cells, such as CD24, KRT8, and KRT18, (ii) being capable of coping with hypoglycemia, acidity, hypoxia, and/or hyperosmolarity conditions imitating a healthy or moderately degenerated intervertebral disc, and (iii) having the same morphological characteristics as nucleus pulposus cells, such as cytoskeletal deposition and comparatively large intercellular vacuoles, preferably, to have traits of a plurality of groups (i), (ii), or (iii). Further, not all the cells belonging to the cell population necessarily have the same traits uniformly, and trait (iii) above may be seen only in a part of the cell population, for example.

In a typical embodiment of the present invention, the nucleus pulposus cell master regulator transcription factors include at least two selected from the group consisting of T, SOX6, and FOXQ1, or homologs thereof (which may be referred to as “first transcription factor group” in this description). In this typical embodiment, the nucleus pulposus cell master regulator transcription factors preferably include T (or a homolog thereof) and SOX6 (or a homolog thereof), or T (or a homolog thereof) and FOXQ1 (or a homolog thereof), more preferably all of T, SOX6, and FOXQ1.

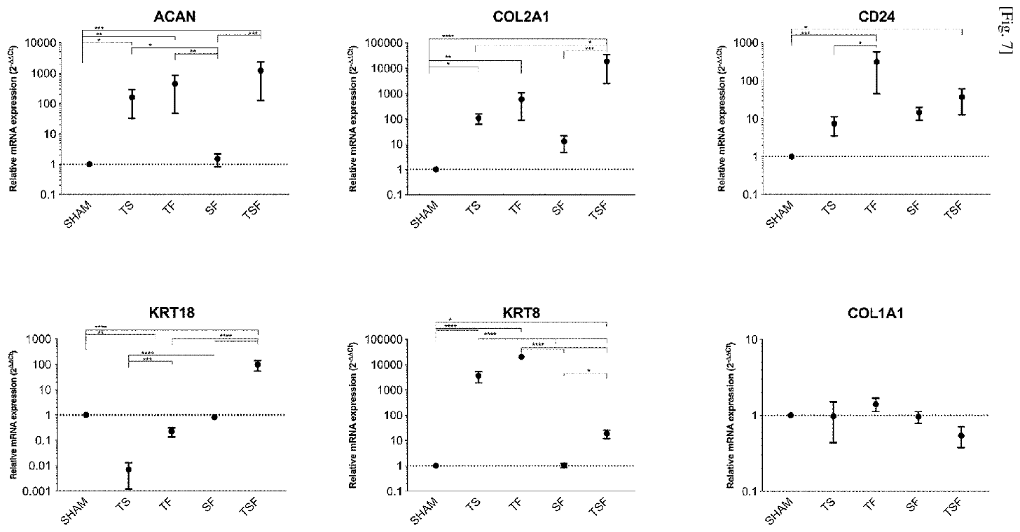

According to one embodiment of the present invention, the master regulator transcription factors preferably include at least one selected from the group consisting of PITX1 and PAX1, or a homolog thereof (herein referred to as “second transcription factor group”) in addition to the aforementioned first transcription factor group. For example, in view of the above (iii) relating to the active nucleus pulposus cell phenotype, PITX1 is preferably added to the master regulator transcription factors in the case of fabricating the induced nucleus pulposus cells by transdifferentiation from fibroblasts or the like in the first embodiment of the present invention (see FIG. 8).

According to one embodiment of the present invention, the master regulator transcription factors preferably include at least one selected from the group consisting of HIF3α, SOX9, RUNX1, HIF1α, and FOXA2, or a homolog thereof (herein referred to as “third transcription factor group”) in addition to the first transcription factor group and/or the second transcription factor group.

According to still another embodiment of the present invention, the master regulator transcription factors preferably include:
T or a homolog thereof; and
at least one selected from the aforementioned transcription factor groups of the present invention excluding T, SOX6, and FOXQ1, or a homolog thereof.

The nucleus pulposus cell master regulator transcription factors of the present invention may be introduced into target cells in the form of a gene (nucleic acid) or in the form of a protein that is a product of the gene. A variety of means for introducing the gene (nucleic acid) or protein into the target cells are known to those skilled in the art, and suitable means and conditions corresponding to the mean can be used in the present invention. The gene (nucleic acid) of the nucleus pulposus cell master regulator transcription factors may be, for example, in the form of DNA such as plasmid or in the form of RNA such as mRNA, and the gene can be introduced into the target cells in each form, for example, by transfection using a composite with a liposome, lipid particles, a polymer, or the like; electroporation; or a viral vector using retrovirus, lentivirus, adeno-associated virus, adenovirus, Sendai virus, or the like. Further, a protein of the nucleus pulposus cell master regulator transcription factors can be introduced into the target cells, for example, by coupling cell penetrating peptides with the protein.

According to one embodiment of the present invention, the nucleus pulposus cell master regulator transcription factors are introduced into the target cells in the form of an expression vector (such as a viral vector plasmid and an expression plasmid) into which a gene encoding at least one of the nucleus pulposus cell master regulator transcription factors has been inserted. The gene on the vector is expressed within the target cells, thereby producing a protein of the nucleus pulposus cell master regulator transcription factor, as a result of which, gene expression specific to nucleus pulposus cells such as aggrecan, type II collagen, and CD24 is directly or indirectly induced. The vector may be an embodiment of existing in the nucleus or cytoplasm either temporarily or continuously while being replicated, or may be an embodiment of permanently existing in the genomic DNA while being incorporated therein. In the case where a plurality of nucleus pulposus cell master regulator transcription factors are expressed, one of the nucleus pulposus cell master regulator transcription factors may be expressed by one expression vector (using a plurality of such expression vectors in combination), or the plurality of nucleus pulposus cell master regulator transcription factors may be expressed by one expression vector.

For example, plasmid DNAs containing genes encoding T, SOX6, and FOXQ1 may be introduced into human newborn dermal fibroblasts by transfection using lentivirus, and the factors can be expressed by a 35M cauliflower mosaic virus promoter (pCMV) disposed in the upstream of the genes. This causes overexpression of the T, SOX6, and FOXQ1 genes in the human newborn dermal fibroblasts having the three nucleus pulposus cell master regulator transcription factors introduced thereinto, so that the cells can be induced into nucleus pulposus cells. The induced nucleus pulposus cells thus obtained are characterized by expression and production of nucleus pulposus cell-specific markers such as aggrecan, type II collagen, and CD24.

According to one embodiment of the present invention, the nucleus pulposus cell master regulator transcription factors of the present invention function as markers of a mature active nucleus pulposus cell phenotype. The nucleus pulposus cell master regulator transcription factors are rarely expressed in the cell population mainly composed of nucleus pulposus cells that have aged and differentiated into a fibrous cell type, such as a degenerated intervertebral disc. In contrast, the expression levels in the cell population mainly composed of healthy nucleus pulposus cells are high. Therefore, the expression levels of the nucleus pulposus cell master regulator transcription factors in a cell population that contains isolated nucleus pulposus cells or nucleus pulposus cells collected from a tissue such as a nucleus pulposus and an intervertebral disc are measured, thereby enabling indices of the nucleus pulposus cell maturity, the cell activity, and the health of the cell population or the tissue to be obtained as indices associated with the aging, degenerating or disease state of the nucleus pulposus cells or the cell population.

### Method for Producing Induced Nucleus Pulposus Cells

The method for producing induced nucleus pulposus cells of the present invention includes at least the steps of introducing the differentiation inducer of the present invention (an effective amount of a gene of nucleus pulposus cell master regulator transcription factors or a product thereof) into cells in vitro (introduction step); and differentiating the cells into an active nucleus pulposus cell phenotype through culturing the cells (differentiation induction step), and preferably further includes the step of testing whether the cells during the differentiation induction step or after the differentiation induction step express at least one selected from the group consisting of CD24, aggrecan, and type II collagen (test step).

### Introduction Step

The introduction step is a step of introducing the differentiation inducer of the present invention (an effective amount of a gene of nucleus pulposus cell master regulator transcription factors or a product thereof) into cells.

The cells as targets for introduction of the differentiation inducer (herein referred to as “introduction target cells”) are not specifically limited, as long as they are nucleated cells other than active nucleus pulposus cell phenotypes, preferably, somatic cells (cells other than germ cells), and include various cell types. The introduction target cells may be an established cell line or may be primary cultured cells (autologous cells or allogeneic cells) collected from an individual or their passage cells.

In the first embodiment of the present invention, the introduction target cells are various somatic cells that have terminally differentiated into cells other than nucleus pulposus cells, or cells that have already been committed to differentiation into cells other than nucleus pulposus cells (progenitor cells of a particular lineage other than nucleus pulposus cells) and are, for example, skin tissue cells that are relatively easy to obtain and culture, typically preferably, fibroblasts.

In the second embodiment of the present invention, the introduction target cells are stem cells such as embryonic stem cells (ES cells) and induced pluripotent stem cells (iPS cells) possessing pluripotency, various stem cells possessing multipotency into nucleus pulposus cells and other cells, or other cells that have not been committed to differentiation into nucleus pulposus cells and are, for example, preferably mesenchymal stem cells (MSC), which are adult stem cells that can be collected from tissues such as fat, umbilical cord, synovium, and bone marrow.

The introduction target cells are generally derived from vertebrate animals, typically, mammals and may be derived from humans or non-human mammals. Examples of mammals can include mice, rats, dogs, cats, sheep, and bovines, in addition to humans. In the case where the introduction target somatic cells are primary cultured cells collected from an individual or their passage cells. the individual may be an individual into which induced nucleus pulposus cells are to be transplanted (such as patients of intervertebral disc disorders) or may be an individual different from the aforementioned individual (a healthy individual or a donor).

The introduction target cells may be derived from a vertebrate animal with genetic variation. For example, nucleus pulposus cell master regulator transcription factors are introduced into skin fibroblasts derived from a target (a human or a non-human vertebrate) with genetic variation to produce induced nucleus pulposus cells, thereby enabling analysis of the influence of genetic variation (the role of the gene) on the behavior of nucleus pulposus cells, intervertebral disc development, and other phenomena.

The differentiation inducer used in the introduction step may be used in an effective (necessary) amount for (i) maintaining and inducing transdifferentiation, in the case where the introduction target cells are terminally differentiated cells other than nucleus pulposus cells or the like, from the cells to an active nucleus pulposus cell phenotype, or an effective (necessary) amount for (ii) differentiation induction, in the case where the introduction target cells are undifferentiated cells or the like, from the cells into an active nucleus pulposus cell phenotype. The amount to be used as the transdifferentiation agent (i) and the amount to be used as the differentiation inducer (ii) each vary depending on the embodiment of the present invention, such as the type of introduction target cells, the types of nucleus pulposus cell master regulator transcription factors and selection of whether they are introduced into the cells in the form of a gene (such as expression plasmid and mRNA) or in the form of a protein, further the means (such as a viral vector, electroporation, microinjection, lipofection, coupling of cell penetrating peptides, and other transfection reagents) for introducing them into the cells, and the cell culture conditions. The numerical range thereof cannot be generally determined. Those skilled in the art would be able to adjust and set the amount to be used, for example, using the ratio of the number of induced nucleus pulposus cells with respect to the total number of cells as an indicator, so that an expected purpose can be achieved.

As an indicator, it is conceivable to set the number of viral vectors per cell to a suitable range according to the concept of the multiplicity of viral infection (MOI), when nucleus pulposus cell master regulator transcription factors are introduced into the introduction target cells in the form of genes using viral vectors. As an example, it is conceivable to use a viral vector solution so that about 8 viral vectors are introduced into each cell (equivalent to MOI=8). Since the number of “about 8” above is mentioned as an example, a larger or smaller number may be employed, and the number can be appropriately set and adjusted by those skilled in the art. Likewise, also in the embodiment in which genes of nucleus pulposus cell master regulator transcription factors are introduced into the cells in a form other than viral vectors, such adjustment can be performed so that an appropriate number of genes of nucleus pulposus cell master regulator transcription factors are introduced into each introduction target cell.

Accordingly, the differentiation inducer of the present invention can be configured as a solution containing genes of nucleus pulposus cell master regulator transcription factors prepared so as to have an appropriate “multiplicity of infection” (such as expression vectors introduced into each cell) or a kit therefor, corresponding to the type of introduction target somatic cells, the number of cells, and other culture conditions.

The introduction step may be performed under conditions suitable for introducing an effective amount of a gene of nucleus pulposus cell master regulator transcription factors or a product thereof into introduction target cells, and culturing the cells after introduction. The culture conditions such as components in the culture medium used therefor (such as the basal culture medium, growth factors, other additive components. vectors, and transfection reagents), the culture period, and the atmosphere can be appropriately set by those skilled in the art.

### Differentiation Induction Step

The differentiation induction step is a step of differentiating the cells into an active nucleus pulposus cell phenotype by culturing cells having the differentiation inducer of the present invention (an effective amount of a gene of nucleus pulposus cell master regulator transcription factors or a product thereof) introduced thereinto by the introduction step.

The differentiation induction step may be performed under conditions suitable for culturing until the cells having the nucleus pulposus cell master regulator transcription factors introduced thereinto become an active nucleus pulposus cell phenotype. The culture conditions such as components in the culture medium used therefor (such as the basal culture medium, growth factors, and other additive components), the culture period, and the atmosphere can be appropriately set by those skilled in the art. Further, the culture in the differentiation induction step may be two-dimensional culture (for example, monolayer culture) or may be three-dimensional culture (for example, 3D pellet culture).

According to one embodiment of the present invention, the culture medium (cell cultures) in the differentiation induction step can contain at least one selected from the group consisting of transforming growth factor β1 (TGFβ1), transforming growth factor β2 (TGFβ2), and transforming growth factor β3 (TGFβ3), and at least one selected from the group consisting of growth differentiation factor 5 (GDF5) and growth differentiation factor 6 (GDF6). In some embodiments of the present invention, it may be preferable, for example, to culture the cells having the differentiation inducer introduced thereinto within a culture medium supplemented with transforming growth factor β1 (TGFβ1) and growth differentiation factor 5 (GDF5), for maintaining and inducing transdifferentiation into an active nucleus pulposus cell phenotype. The concentration of each of TGFβ1 and GDF5 in the culture medium can be appropriately adjusted, but the concentration of TGFβ1 is generally 1 to 10,000 ng/mL, preferably 10 to 100 ng/mL, for example, about 10 ng/mL, and the concentration of GDF5 is generally 1 to 100,000 ng/mL, preferably 10 to 500 ng/mL, for example, about 100 ng/mL.

Further, the culture medium in the differentiation induction step can contain dexamethasone at a concentration of generally 1 to 1,000 ng/mL, preferably 4 to 500 ng/mL, for example, about 10 ng/mL. The culture medium in the differentiation induction step can contain L-ascorbic acid at a concentration of generally 1 to 1.000 μM, preferably 5 to 500 μM, for example, about 50 μM.

According to one embodiment of the present invention, the differentiation induction step can include culturing cells under at least one condition selected from the group consisting of a hypoxic environment, an acidic environment, and a low-glucose environment, more preferably under all these conditions. Induced nucleus pulposus cells which are viable in a hypoglycemic, acidic, and hypoxic (and hyperosmolar state) environment that is the environment in a healthy or moderately degenerated intervertebral disc can be fabricated and recovered by culturing cells having nucleus pulposus cell master regulator transcription factors introduced thereinto in such an environment. The hypoxic environment generally refers to an environment in which the oxygen concentration in the atmosphere of the culture medium is 1 to 10%, preferably 1 to 5%, for example, about 2%. The acidic environment generally refers to an environment in which the pH of the culture medium at room temperature (for example, 25° C.) is in the range of 6.5 to 7.4, for example, about 6.8. The low-glucose environment generally refers to an environment in which the glucose concentration in the culture medium is 4.5 g/L or less, for example, about 1 g/L. The culture period under such an environment can be appropriately adjusted but is generally 2 to 90 days (3 months), for example, 14 days (two weeks). For example, it is preferable to culture cells having with nucleus pulposus cell master regulator transcription factors introduced thereinto under a hypoxic environment with an oxygen concentration of 2% for two weeks.

### Checking Step

In the checking step, the expression status of a gene or a protein that characterizes induced nucleus pulposus cells, that is, a nucleus pulposus cell marker in the cells during culture or after culture in the differentiation induction step is checked. As such a nucleus pulposus cell marker, either a marker that is positive in an active nucleus pulposus cell phenotype (positive marker) or a marker that is negative therein (negative marker) can be used. The expression status of a gene and/or a protein serving as a nucleus pulposus cell marker can be checked quantitatively or qualitatively, for example, by a general approach such as real time polymerase chain reaction (real time PCR), immunohistological staining (IHC), Western blotting, and flow cytometry. Whether the expression is positive or negative can be determined based on the results.

Examples of the positive marker in induced nucleus pulposus cells include CD24, aggrecan, type II collagen, keratin 8, and keratin 18. According to one embodiment of the present invention, the cells during culture or after culture in the differentiation induction step, that is, (the cells estimated to be) induced nucleus pulposus cells preferably express (being positive in) at least one selected from the group consisting of CD24, aggrecan, and type II collagen, more preferably express (being positive in) all these three types.

Examples of the negative marker in induced nucleus pulposus cells include type I collagen. According to one embodiment of the present invention, the cells during culture or after culture in the differentiation induction step, that is, (the cells estimated to be) induced nucleus pulposus cells preferably do not express (being negative in) or weakly express type I collagen.

Transdifferentiation or differentiation induction from the cells into an active nucleus pulposus cell phenotype is paired with a decrease in proliferative capacity, thereby achieving differentiation and maturity of the cells having master regulator transcription factors introduced thereinto. When expression of such a nucleus pulposus cell-specific marker in the growth factor introduction cells (the growth factor introduction cells contained in a cell population in a cell culture at a desired ratio) during culture is confirmed in the test step, the production of induced nucleus pulposus cells can be ended.

The method for producing induced nucleus pulposus cells of the present invention as described above enables an active nucleus pulposus cell phenotype to be supplied substantially infinitely (inexhaustibly). Use of induced nucleus pulposus cells to be obtained by the present invention is not limited, but the cells can be used typically for administration to an intervertebral disc in methods for treating and preventing the intervertebral disc disorders described below, particularly, for preparing cell preparations used in such applications.

### Transcription Factor Introduction Cells/Induced Nucleus Pulposus Cells

Both the transcription factor introduction cells and induced nucleus pulposus cells of the present invention are cells produced by the method for producing induced nucleus pulposus cells of the present invention. In this description, cells containing an effective amount of a gene of nucleus pulposus cell master regulator transcription factors or a product thereof, that is, cells just having master regulator transcription factors introduced thereinto, and cells that overexpress nucleus pulposus cell master regulator transcription factors due to culture after the introduction but are not yet induced into an active nucleus pulposus cell phenotype are referred to as “transcription factor introduction cells”, whereas cells that are obtained by culturing the transcription factor introduction cells and induced into an active nucleus pulposus cell phenotype are referred to as “induced nucleus pulposus cells”, so as to distinguish the two. The embodiment of a cell population containing transcription factor introduction cells and/or induced nucleus pulposus cells, and the embodiment of a cell culture containing such a cell population can be adjusted corresponding to their applications. For example, in the case where a cell population containing induced nucleus pulposus cells is used as a raw material for producing a cell preparation for treating or preventing an intervertebral disc disorder, it is desirable to set the ratio of induced nucleus pulposus cells in the cell population as high as possible (conversely, to set the ratio of transcription factor introduction cells that have not transformed to induced nucleus pulposus cells as low as possible).

Further, according to one embodiment of the present invention, inducer introduction cells, cell cultures, induced nucleus pulposus cells, or cell populations obtained by the present invention can be used in a method for screening for a medicine or a method for treating or preventing an intervertebral disc disorder in a vertebrate animal including a step of testing the effectiveness and safety in a subject, that is, can be used as an in-vitro test model serving as a scientific, diagnostic, or prognostic tool for evaluating the reaction of nucleus pulposus cells or patient-specific nucleus pulposus cells to dosing, factors, or other (environmental) conditions. Further, a methodology established by the present invention can be used, for example, for fabricating nucleus pulposus cells from patients with genetic defects, in order to evaluate the influence of the genetic defects on nucleus pulposus cell phenotypes, homeostasis, development and pathology of an intervertebral disc, and reactions to drugs. The embodiment based on such applications, for example, enables the effectiveness of specific drugs on individual patients to be evaluated before administration, or the effectiveness of a treatment for preventing toxic side effects in administration of induced nucleus pulposus cells to patients or suppressing medical cost to be evaluated, in an individualized medical approach.

### Pharmaceutical Composition and Cell Preparation

The pharmaceutical composition of the present invention contains the differentiation inducer of the present invention, that is, an effective amount of a gene (nucleic acid) of nucleus pulposus cell master regulator transcription factors or a product thereof (protein). Further, the cell preparation of the present invention contains induced nucleus pulposus cells obtained by introducing an effective amount of nucleus pulposus cell master regulator transcription factors into cells outside the body (in vitro or ex vivo). The pharmaceutical composition and the cell preparation can be used for treating and preventing spine-related diseases of humans and non-human vertebrates. The pharmaceutical composition of the present invention can be used in the form of so-called gene treatment for transforming cells present in the nucleus pulposus into an active nucleus pulposus cell phenotype inside the body (in vivo or in situ). Meanwhile, the cell preparation of the present invention can be used as an effective supply source, which potentially recovers biomechanical characteristics of the spine, for reconstituting and recovering the structure of an intervertebral disc by being transplanted into a nucleus pulposus with a degenerated intervertebral disc through allogeneic transplantation, xenotransplantation, or autologous cell transplantation.

The dosage forms of the pharmaceutical composition and the cell preparation of the present invention need only to enable delivery to a nucleus pulposus of an intervertebral disc serving as a target but can be, for example, injections, preferably, injections for local administration to the intervertebral disc (nucleus pulposus). The pharmaceutical composition and the cell preparation, for example, when prepared as injections, can contain pharmaceutically acceptable substances such as injection solvents, normal saline, culture liquids, other suitable solvents/dispersion media, additives, and the like, as required. Further, the pharmaceutical composition and the cell preparation each can be produced also as a kit including a syringe and a pharmaceutical agent to be used in combination, as required.

### Method for Treating and Preventing Intervertebral Disc Disorder

A first embodiment (which may be referred to as “first treatment and prevention method” in this description) of the method for treating and preventing an intervertebral disc disorder of the present invention includes transplanting or administering (a cell population containing) the induced nucleus pulposus cells of the present invention or the cell preparation of the present invention containing the induced nucleus pulposus cells (cell population) in vivo so as to act on the intervertebral disc nucleus pulposus tissue. The phrase “so as to act on the intervertebral disc nucleus pulposus tissue” means that the embodiment of transplantation or administration is not specifically limited, as long as the induced nucleus pulposus cells or the like that have been transplanted or administered can reach the intervertebral disc nucleus pulposus tissue that is an affected area, so that the effects of treatment or prevention can be exerted. The phrase includes transplanting the induced nucleus pulposus cells or the like into the intervertebral disc nucleus pulposus tissue or the vicinity thereof or injecting the induced nucleus pulposus cells or the like so as to reach the affected area through blood vessels, for example.

A second embodiment (which may be referred to as “second treatment and prevention method” in this description) of the method for treating and preventing an intervertebral disc disorder of the present invention includes administering the differentiation inducer of the present invention or the pharmaceutical composition of the present invention containing the differentiation inducer in vivo so as to act on nucleus pulposus cells in an intervertebral disc. The phrase “so as to act on nucleus pulposus cells in an intervertebral disc” means that the embodiment of administration is not specifically limited, as long as the administered differentiation inducer or the like can be incorporated into the nucleus pulposus cells in the intervertebral disc to reactivate the cells, so that the effects of treatment or prevention can be exerted. The phrase includes administering the differentiation inducer or the like into the intervertebral disc nucleus pulposus tissue or the vicinity thereof in situ or injecting the differentiation inducer or the like so as to reach the affected area through blood vessels, for example.

The intervertebral disc (nucleus pulposus tissue) that is subjected to the action of the cell preparation of the first treatment and prevention method of the present invention or the like and the pharmaceutical composition or the like of the second treatment and prevention method is an intervertebral disc with degeneration, aging, other symptoms. In such an intervertebral disc with degeneration or the like, healthy nucleus pulposus cells decrease, and senescent cells and fibrous nucleus pulposus cells increase. The induced nucleus pulposus cells transplanted or administered by the first treatment and prevention method and the induced nucleus pulposus cells generated (reactivated) by the second treatment and prevention method can survive in an intervertebral disc microenvironment, which is acidic, hyperosmotic and hypoglycemic, thereby increasing the amount of aggrecan, type II collagen, and the like to be produced in the extra-cellular matrix, so that the intervertebral disc disorder can be treated or prevented. In the second treatment and prevention method of the present invention, nucleus pulposus cell master regulator transcription factors are introduced into senescent cells and fibrous nucleus pulposus cells contained in the intervertebral disc, so that those cells can be transdifferentiated and induced into an active nucleus pulposus cell phenotype.

The cell preparation or the like of the first treatment and prevention method of the present invention and the pharmaceutical composition or the like of the second treatment and prevention method may be administered in an effective amount for exerting desired treatment or prevention effects. Such an effective amount can be appropriately adjusted depending on the dose per administration, the number of doses, and the administration interval (the number of doses within a certain period), in consideration of the embodiments of the cell preparation and the pharmaceutical composition or the like, the administration subject, the administration route, and the like. Both the first and second treatment and prevention methods can be performed on humans and non-human vertebrates.

### EXAMPLES

### (1) Human Nucleus Pulposus Tissue

In conducting this study, collection and use of human tissue samples were approved by the institutional ethics review committee of Tokai University Hospital. In addition, surgically excised tissue materials were obtained only from patients which have provided their informed consent.

### (2) Cell Separation and Culture

A human intervertebral disc tissue collected was macroscopically examined to separate the nucleus pulposus tissue from fibrous tissue and other tissue structures. Subsequently. the nucleus pulposus tissue was incised into about 1 cm³ sections. The human nucleus pulposus tissue was further digested with TrypLE express (Gibco, USA) at 37° C. for 1 hour. Thereafter, the first digested tissue was transferred to 0.25 mg/mL collagenase-P (F. Hoffmann-La Roche, Ltd., Switzerland) for 2 hours. The suspension obtained was filtered with a 100 μm cell strainer, washed twice with 10% FBS-added αMem (Dulbecco, USA), and inoculated at a cell density of 3,000 to 5,000 cells/cm². The seeded cells were cultured at 37° C. under 5% CO₂, and 5% O₂ until further use.

### (3) Microarray Assay

The nucleus pulposus cells were seeded at a density of 3,000 to 5,000 cells/cm² and grown with 10% (vol/vol) FBS and αMEM (Gibco) in a wet chamber at 37° C. under 25% O₂. For cell purification, the nucleus pulposus cells were incubated with anti-human disialoganglioside GD2 (GD2) (BD Pharmingen; 14; G2a) mAb for 30 minutes and then with FITC-conjugated anti-mouse Ig goat (BD Biosciences) at 4° C. for 30 minutes. After washing, the cells were stained with allophycocyanin-conjugated anti-human Tie2 (R&D Systems, Inc., clone 83715) mAb and PE-conjugated or biotinylated anti-human CD24 (BD Biosciences; clone ML5) mAb for 1 hour. The cell suspension was centrifuged at 4° C. and 1200 rpm for 5 minutes and washed. Subsequently, the nucleus pulposus cells were divided into Tie2+/GD2+/CD24−, Tie2−/GD2+/CD24−/+, and Tie2−/GD2−/CD24+ populations by FACS Vantage cells (BD Biosciences). At the same time, the Tie2−/GD2−/CD24+ population was also separated from spheroids formed by 3D cell culture, using a methylcellulose culture medium, MethoCult H4230 (STEMCELL Technologies Inc.). Subsequently, the fractionated nucleus pulposus cell population was dissolved in RNA buffer of RNeasy Mini Kit (QIAGEN). Total RNA was isolated. Finally, Cy3-labeled cRNA was prepared from each of the four populations using a low-input Quick Amp Labeling Kit, one color (Agilent Technology, Inc.), analyzed using SurePrint G3 Human GE 8x60K v2 Microarray (Agilent Technology, Inc.) and Genc Expression Hybridization Kit (Agilent Technology, Inc.), and analyzed by Agilent DNA Microarray Scanner (G2565CA) using a software, Feature Extraction 7 (Agilent Technology, Inc.). The expression values obtained were compared with the expression levels in neural progenitor cells (ID GSM1608144, GSM1608145), fibroblasts (ID GSM1191059, GSM1191060, GSM1191061), iPS cells (ID GSM1598135, GSM1598136, GSM1598137), and lung cells (ID GSM1700910, GSM1700913) obtained from the NCBI database (https://www.ncbi.nlm.nih.gov/gds/) by subtraction.

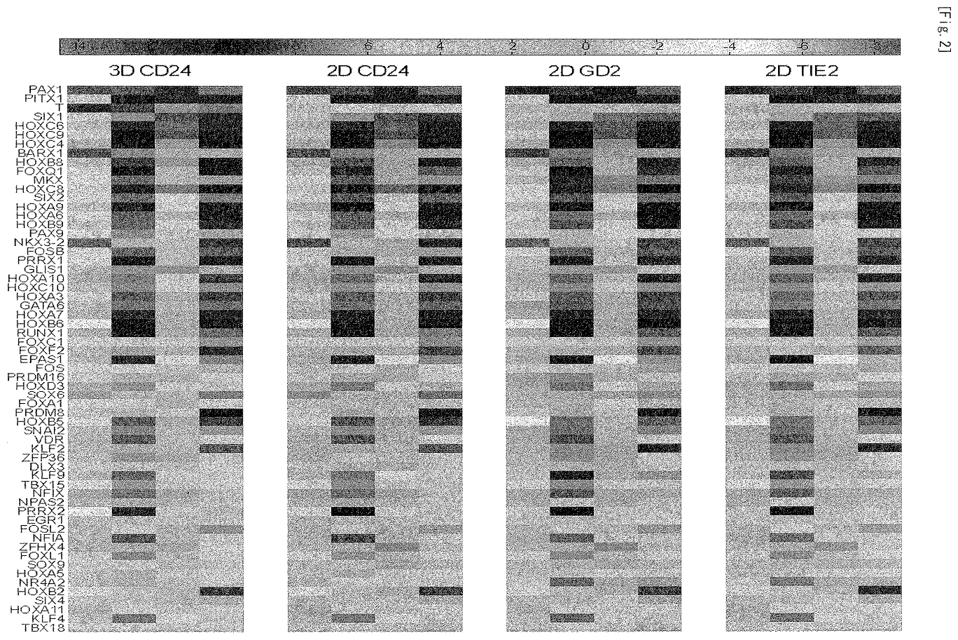

FIG. 2 shows the results. Microarray assays revealed 131 distinct transcription factors showing relatively high expression in human nucleus pulposus cells as compared with the human neural progenitor cells, the human lung cells, the human-induced pluripotent stem cells, and the human dermal fibroblasts. These transcription factors arranged in descending order of relative expression ratio are as follows: PAX1, PITX1, BARX1, FOXQ1, HOXC9, HOXC4, HOXA9, HOXA6, HOXB8, PAX9, PRRX1, FOSB, HOXB9, HOXA3, HOXA10, HOXC6, GLIS1, EPAS1, SIX1, MKX, HOXC8, GATA6, NKX3-2, SIX2, RUNX1, HOXA7, HOXB6, HOXC10, FOXC1, HOXA5, HOXD3, FOXF2, FOS, ZFP36, FOXL1, KLF9, SNA12, TBX15, KLF2, NFIA, PRRX2, KLF4, HOXB5, PRDM8, T, VDR, HOXA2, HOXA11, HOXB3, NR4A2, FOXF1, FOSL2, NFIX, TLE2, NR4A3, NPAS2, HSF4, SIX4, HOXB7, HOXB2, EGR1, KLF8, ZMAT3, ZFHX4, MYC, SOX9, NFIL3, STAT2, FOXC2, GATA3, PRDM16, SMAD9, SNA11, JUNB, HOXD8, DMRTA1, SOX6, PITX2, PARG, FOXS1, FLI1, FOSL1, NFATC4, FOXA1, MAFB, DLX3, SOX5, NR3C1, PKNOX2, VAX1, LBX2, HOXB4, OSR1, THZ3, DLX5, MEIS2, GSC, KLF5, MSC, TBX18, KLF10, FOXP2, GLIS3, DBX1, HOXC12, DLX2, HOXC5, PLAGL1, HOXC13, HOXD10, PAX6, HNF4A, NKX6-1, ZBTB7B, HLF, TBX4, MLXIPL, NFATC1, ID3, ATF3, ID2, HOXC11, HIVEP2, PGR, PPARA, ZIC1, MYBL1, LHX9, OSR2, STAT1, FEY, TBX2, TWIST1, STAT6, MAF, STAT5A, PAX8, EGR2, TOX, EBF1, FOXD4, OTX1, NFE2L3, HOXD9, ZEB1, NKX3-1, TSHZ2, ELF4, FOXP1, EBF3, DLX4, TBX1, KLF6, SIX5, TSHZ1, HOXA4, FOX01, RUNX2, DMRT1, IRX3, ETS1, MXD4, ZHX1, HNF1A, MEIS1, RUNX3, HAND2, EGR3, VAX2, MEOX2, HIF1 A, BNC1, ZFPM2, HIC1, MSX1, SHOX2, JUN, CREB3L 1, TBX3, HOXA 13, BACH2, 1D1, STAT4, SOX11, IRX5, HESS, LHX1, BCL6B, NR2F2, NR2F1, NANOG, FOXA2, and FOXM1.

### (4) Plasmid Amplification

A plasmid with transgenes for all nucleus pulposus cell master regulator transcription factor candidates inserted into the pMXs-GW skeleton, and a plasmid with green fluorescent protein (GFP), additionally Brachyury (T) was also inserted into the pMX-IRES-GFP skeleton, were used. The plasmids were provided by courtesy of the iPS cell research and use center of Kyoto University in Japan. Plasmid amplification was established by cloning each plasmid into MAX Efficiency Stb12 chemically competent cells (Invitrogen, USA) through heat shock at 42° C. for 45 seconds and subsequent overnight incubation in LB-broth (Miller) liquid microbial growth medium (Sigma-Aldrich Co. LLC, USA). On the next day, plasmid DNA was recovered using the NucleoBond Xtra Midi kit (Takara Bio Inc., Japan) according to the manufacturer's instructions.

### (5) Virus Particle Production

Virus particles were produced by separate transfection of the plasmid DNA obtained according to research previously published by Kitazawa et al. (Kitazawa. K. et al. OVOL2 Maintains the Transcriptional Program of Human Corneal Epithelium by Suppressing Epithelial-to-Mesenchymal Transition. Cell reports 15, 1359-1368, doi:10.1016/j.celrep.2016.04.020 (2016)). In summary, platinum-GP retroviral packaging (PLAT-GP) cells in a DMEM high glucose culture medium supplemented with 10% FBS, 1% pyruvate, and 50 U/mL (50 μg/mL) penicillin/streptomycin were seeded at a density of 55×10³ cells/cm² on a plastic coated with 0.1% gelatin (Sigma-Aldrich Co. LLC, USA) derived from pig skin. On the next day, 5.4 μL of FuGENE (registered trademark, Promega KK., USA), 600 ng (0.6 μL) of pLP/VSVG (registered trademark, Invitrogen, USA) (expression plasmid of vesicular stomatitis virus G protein as an alternative envelope), 1200 ng (1.2 μL) of pMXs-GW plasmid or pMX-IRES-GFP vector DNA encoding a singular transgene, and 60 μL of Opti-MEM (registered trademark, Thermo Fisher Scientific, Inc., USA), which account for a total of 69 μL, were added per 2 mL of PLAT-GP culture medium. PLAT-GP cells were incubated in a wet chamber at 37° C. under 21% O₂ for 24 hours, and thereafter the culture medium was refreshed. After further culture for 24 hours, the culture medium was recovered and filtered with a 0.45 μm pore size filter unit (Sigma-Aldrich Co. LLC, USA). The virus-containing culture medium was directly applied.

### (6) iPS Cells Interference Assay

According to research by Takahashi et al. (Takahashi, K. and S. Yamanaka, Induction of pluripotent stem cells from mouse embryonic and adult fibroblast cultures by defined factors. Cell, 2006. 126 (4): p. 663-76.), mouse SNL feeder cells for iPS cell culture were seeded on 0.1% gelatin-coated wells and grown to 70 to 80% confluence.

Meanwhile, the nucleus pulposus cells were seeded at 555 cells/cm² on 6-well plate wells. Next, the culture medium was replaced with DMEM high glucose (Thermo Fisher Scientific, Inc.) supplemented with 10 ng/ml of polybrene (Santa Cruz Biotechnology, Inc., USA) and 1% penicillin/streptomycin (Thermo Fisher Scientific, Inc., USA). Subsequently, 2 mL of a mixed culture medium rich in VSVG particles containing the plasmid DNA encoding the transgene was added thereto at maximum. Viral transduction was promoted by centrifuging the nucleus pulposus cultures at 800 G and 35° C. for 30 minutes. Subsequently, the culture medium was aspirated and was replaced with 2 mL of DMEM high glucose culture medium supplemented with 10% FBS and 1% penicillin/streptomycin. Transduction was performed using a viral vector (pMX-GW) containing genes for Yamanaka factors (OCT3/4, SOX2, KLF4, and CMYC) and additional master regulator transcription factor candidates (see the microarray data above). The nucleus pulposus cells were recovered under 2% O₂ hypoxia and tension for 48 hours with the culture medium replaced everyday.

Two days later, the SNL feeder cells were treated with 13 μg/mL of mitomycin C (FUJIFILM Wako Pure Chemical Corporation, Japan) for 1 hour. Subsequently, the culture medium was replaced with 10% FBS (Thermo Fisher Scientific, Inc.) and 1% penicillin/streptomycin and refreshed. The nucleus pulposus cells were trypsinized and seeded on the mitomycin C-treated SNL feeder cells. Further, the nucleus pulposus cultures were switched to 21% O₂ every other day, and replacement of the culture medium was also conducted.

Six days after transduction, the culture medium was replaced with ReproCELL primate ES cell culture medium (ReproCell Inc., Japan) supplemented with 10 ng/ml of fibroblast growth factor (FGF) and 1% (v/v%) penicillin/streptomycin. After iPS cell induction was continued for a total of two weeks, the cultures were fixed and stained with alkaline phosphatase staining kit (MUTO pure chemicals Co., Ltd., Japan) according to the manufacturer's instructions. Positively stained colonies were manually counted as an indicator of iPSC induction efficiency.

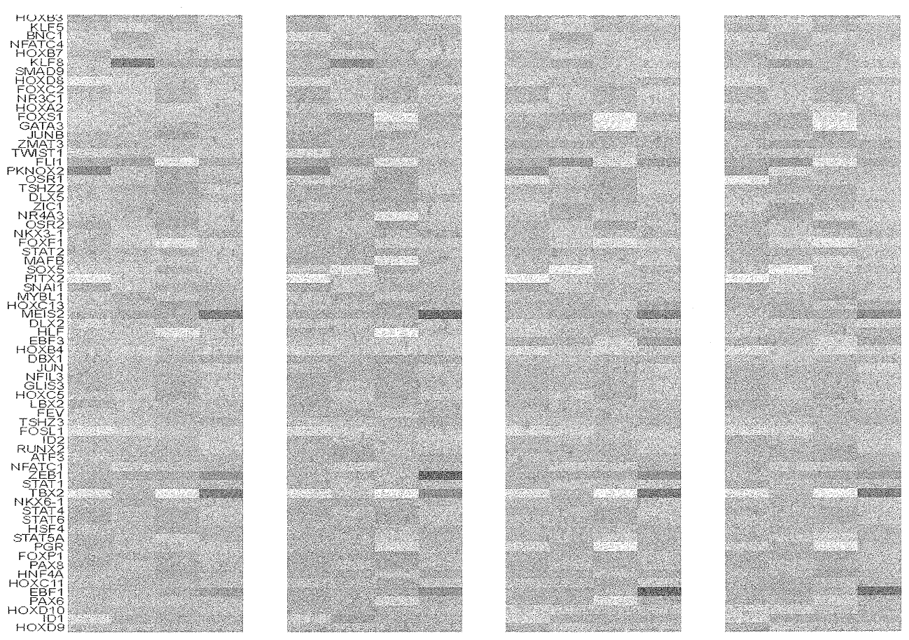

FIG. 3 shows the results. The transcription factors targeted in the microarray assay were each combined with the four Yamanaka factors (OCT3/4, c-MYC, SOX2, and KLF4), and 86 of the transcription factors prevented induction into iPS cells. In particular, complete inhibition of iPS cell colony formation was observed by combining any one of 8 transcription factors, PITX1, T, HOXC9, EBF1, NFIX, NFIA, KLF6, and KLF9 with the Yamanaka factors.

### (7) siRNA-Mediated Interference Assay

In order to examine how each master regulator transcription factor candidate identified by the microarray assay (the results will be described later) affects nucleus pulposus cell marker expression by RNA interference (RNA i), nucleus pulposus cells seeded at 15,000 cells/cm² were transduced with DharmaFECT and custom-made siRNA molecules (GE Healthcare, USA) targeting the mRNA of each master regulator transcription factor candidate, according to the manufacturer's instructions. After transduction with siRNA, the cells were cultured at 37° C. under 2% O₂ in αMem supplemented with 10% FBS for 48 hours.

The cells after culture were collected, and the total RNA was recovered according to the manufacturer's protocol using SVTotal RNA Isolation System (Promega KK., USA). The total RNA recovered was converted into cDNA according to the manufacturer's protocol using High Capacity cDNA Reverse Transcription kit (Applied Biosystem, USA). The mRNA expression level was evaluated from cDNA by 7300RT-PCR system (Applied Biosystems, USA) using SYBR Green PCR master mix (Applied Biosystems) and a primer manually designed for each nucleus pulposus cell marker. CT values obtained by such a system were normalized to the CT value of glyceraldehyde 3-phosphate dehydrogenase (GAPDH; housekeeping gene) and were then compared with the CT value of nucleus pulposus cell sham control (SHAM control) normalized in the same manner, thereby calculating relative values of the expression level.

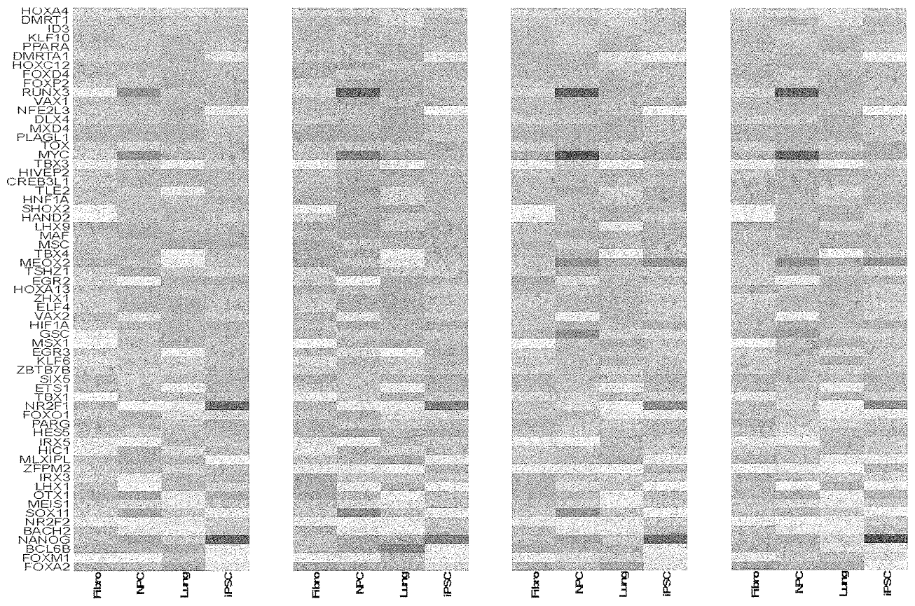

FIG. 4 shows the results. Introduction of siRNA targeting any one of EPAS1, FOXQ1, PITX1, SOX9, SOX6, RUNX1, HIF1α, HIF3α, FOXA2, and SIX1 into the nucleus pulposus cells caused a significant consistent reduction in expression level in almost all sets of nucleus pulposus cell markers evaluated, and the importance for maintaining the nucleus pulposus cell phenotype of the gene was confirmed. Conversely, siRNA targeting PAX1, PAX9, HOXc9, PRDM8, and FOXF1 showed a tendency to increase nucleus pulposus cell marker expression levels, and only limited nucleus pulposus cell markers showed a reduction in expression level.

### (8) Transdifferentiation of Human Fibroblasts: Part 1

Human newborn skin fibroblasts (Lonza, Switzerland) were grown in DMEM containing 10% (v/v) FBS, 100 U/mL penicillin, and 100 μg/mL streptomycin. The day before transduction, human fibroblasts were separated by incubation in 0.25% (w/v) trypsin, 0.001% (w/v) ethylenediaminetetraacetic acid (EDTA), and PBS for 5 minutes and then seeded at a density of 5.5×10³ cells/cm² in a well plate sufficiently covered with DMEM containing 6100 μg/mL penicillin and 100 μg/mL streptomycin.

To each well, 500 μL of viral culture medium transduced with each transgene was added in a maximum volume of 2 ml. As the SHAM control, a GFP transgene vector was added. DMEM containing 100 U/mL of penicillin and 100 μg/mL of streptomycin was added in a total capacity per well of 4 ml. Further, the culture medium was supplemented with 40 μg of polybrene (Santa Cruz Biotechnology, Inc.). The cultures were spun down at 30° C. and about 800 G for 30 minutes. Subsequently, the virus-containing culture medium was removed, and the cells were washed with excess PBS. Finally, the cells were cultured at 37° C. under 2% O₂ for two weeks in DMEM containing 0.1% (v/v) insulin-transferrin-selenium-ethanolamine solution (ITS-X; Thermo Fisher Scientific, Inc.), 1% (v/v) FBS, 50 μM L-magnesium ascorbyl phosphate n-hydrate (FUJIFILM Wako Pure Chemical Corporation, Japan), 100 U/mL penicillin and 100 μg/mL streptomycin, with or without supplementation with 10 ng/mL of TGFβ1 (PeproTech, Inc., Japan) and 100 ng/mL of growth differentiation factor 5 (GDF5; PeproTech. Inc.), and a fresh culture medium was given every 3 to 4 days. The cell morphology was captured by an optical microscope, and temporal changes were revealed depending on the combinations of transcription factors used for transduction.

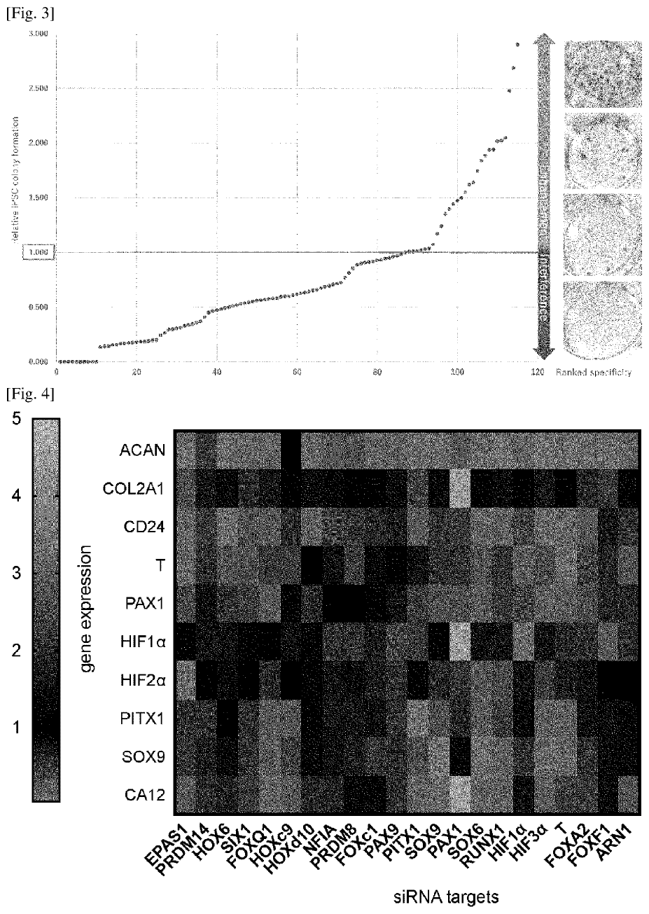

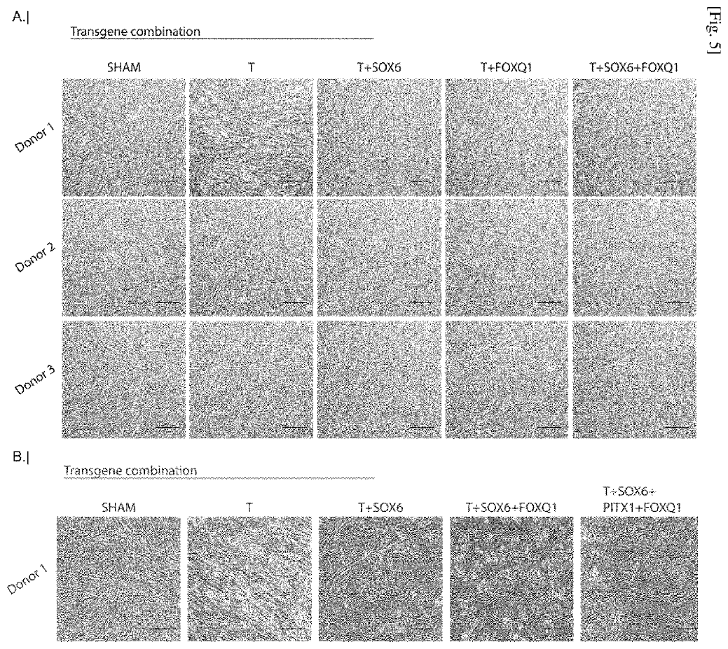

FIG. 5 and FIG. 6 show the results. Untreated, SHAM, or T-transduced fibroblasts showed little change while maintaining their elongated morphology throughout the differentiation culture (FIG. 5A). In contrast, transduction with a combination of two or three selected from T, SOX6, and FOXQ1 consistently resulted in a heterogeneous population with small fractions of cells maintaining the morphology of fibroblasts. Further, it seems from microscopic observation that the size of the cells increases, and the growth tendency is lost. Morphologically, the cells could be divided into three subpopulations: (i) cells having an elongated cell shape with the presence of long processes from the center, (ii) polygonal cells presenting strong cytoskeletal deposition, and (iii) astrocytic morphology presenting dendritic processes (FIG. 5A).

Finally, cells of a group transduced with transcription factors in combination sporadically presented cells with intercellular vacuoles similar to vacuoles observed in notochord cells in vitro (FIG. 6). Likewise, addition of PITX1 (data not shown) was accompanied by strong cytoskeletal deposition and increased cell size, strongly increasing the phenotype of (ii) above.

### (9) Transdifferentiation of Human Fibroblasts: Part 2

The transduced cells were collected with 0.25% trypsin and 0.001% EDTA and were subjected to further evaluation. For samples used for qPCR evaluation, the total RNA was isolated as described above (see (8) siRNA-mediated interference assay). The isolated RNA was thereafter converted into cDNA using a high-volume RNA-to-cDNA kit (Thermo Fisher Scientific, Inc.) according to the accompanying instructions. Subsequently, about 10 to 100 ng of cDNA was used for SYBRGREEN (Thermo Fisher Scientific, Inc.)-mediated qPCR analysis, and a custom-designed primer set for nucleus pulposus markers and notochord markers was applied. Each expression level obtained was calculated as a value of 2^{-ΔΔCT} comparing the gene expression level with that of GAPDH and subsequent SHAM control.

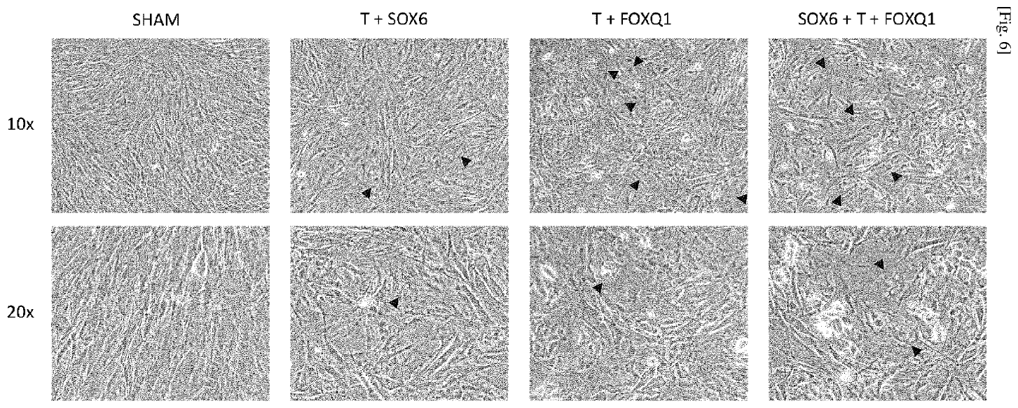

FIG. 7 shows the results. The qPCR analysis revealed a clear tendency of an increase in nucleus pulposus cell marker expression in the cells transduced with a combination of master regulator transcription factors identified, as compared with that in the SHAM-transduced cells. The expression profiles obtained showed an increase in mRNA expression levels of several selected nucleus pulposus cell markers for all combinations of double or triple transcription factors, T, FOXQ1, and SOX6 in fibroblasts one week after the transduction. These results revealed that transduction with a combination of T, SOX6, and FOXQ1 caused the most potent and the most consistent tendency to increase the extracellular matrix and the mRNA levels of aggrecan (ACAN) and type II collagen (COL2). Further, T+SOX6 and T+FOXQ1 showed a relatively higher KRT8 expression level as compared with the SHAM control, and transduction with triple transcription factors showed a strong tendency to increase the nucleus pulposus cell markers, KRT18 and KRT8. Further, most combinations showed a strong increasing tendency in CD24, and the combinations of T+SOX6+FOXQ1 and T+FOXQ1 were the highest. Further data (not included herein) showed that TSF and other combinations tend to improve the expression levels of PITX1, ANXA3, and OVOS. Finally, a negative marker, COL1A1, showed a desirable decreasing tendency only for T+SOX6+FOXQ1.

### (10) Transdifferentiation of Human Fibroblasts: Part 3

Further, two weeks after monolayer differentiation culture, transduced fibroblasts were counted as above at a density of 250,000 cells put into a 15 ml polypropylene conical tube (BD Biosciences) in 0.5 ml of the aforementioned differentiation culture medium. The cell suspension was spun down at 1500 rpm for 5 minutes at room temperature. After the cell pellets obtained were cultured for one day, the pellets were gently tapped from the bottom of the conical tube, and the spherical cell aggregates were further cultured under 2% O₂ for three weeks. Finally, the pellets were fixed with 4% (v/v) paraformaldehyde, supplemented with Tissue-TEK O.C.T. compound (Sakura Finetek Japan Co., Ltd., Japan), and rapidly frozen in liquid nitrogen. The sample obtained was cryosectioned into an 8 μm section on a silane-coated slide (MUTO pure chemical substance). Subsequently, production of ECM within a pellet was visualized by staining the section with a 1 g/L Safranin-O (Merck, USA) and 800 mg/L Fast Green FCF staining (Merck) solution or hematoxylin eosin staining.

FIG. 8 shows the results. First, a pellet culture of fibroblasts transduced with GFP (SHAM) or a single transcription factor candidate (data not shown) generated a completely fibrous pellet structure without the presence of specific characteristics of a notochord or nucleus pulposus phenotype, which is shown by the lack of vacuolated cell morphology or the lack of proteoglycan deposition. In contrast, a pellet composed of fibroblasts transduced with a combination of transcription factors selected from SOX6, T, PITX1, and FOXQ1 showed a strong change in cell morphology toward a notochord phenotype and presented large vacuoles in the cytoplasm. Further, a pellet composed of fibroblasts transduced with T+SOX6, particularly T+SOX6+FOXQ1 showed mildly to intensely safranin-O stained regions and showed proteoglycan deposition throughout the notochord cell-like region within the pellet. The appearance of vesicles that are found in notochord cells indicates that differentiation from fibroblasts into a nucleus pulposus cell line was successful. This is because these characteristics cannot be generally seen in other mammalian cell types. Further, the presence of proteoglycans stained with safranin-O shows effective activation of the chondrogenic characteristics of a nucleus pulposus cell phenotype, indicating that there is a tendency of the cells to differentiate into a more mature nucleus pulposus cell phenotype beyond a notochord cell phenotype within the pellet culture.

### (11) Differentiation Induction of MSCs: Part 1

In the same manner as in the protocols of (8) to (10) above, the master regulator transcription factors were used also for MSCs to induce differentiation into a nucleus pulposus cell phenotype, and the differentiation process was investigated from cell morphological changes, mRNA expression changes, and protein production tested by pellet culture based on histology.

FIG. 5 and FIG. 8 show the results for cell morphological changes. Two weeks after the differentiation induction, MSCs of the SHAM maintained the original elongated cell morphology. Most part of cells transduced with T remained elongated, but larger polygonal cells began to appear in the MSC population. Transduction with a combination of two or three transcription factors selected from T, SOX6, and FOXQ1 changed the morphology of MSCs with an increase in cell size, cytoskeletal deposition, and an increase in cell processes, in the same manner as the changes observed in the evaluation of fibroblasts. Further, the cells exhibited a polygonal, elongated, or astrocytic cell shape and presented a heterogeneous population, in the same manner. The addition of PITX1 also resulted in a strong increase in apparent cytoskeleton production and an increase in larger cell types. Further, in the complex-transduced group, some cells within the heterogeneous population contained notochord cell-like vacuoles in their cytoplasm and showed an increase in larger cell types (data not shown).

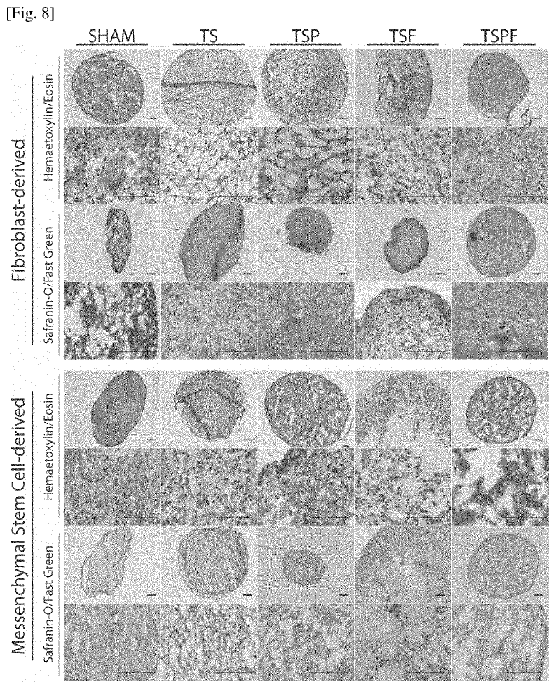

FIG. 9 shows the results for mRNA expression changes. As compared with the SHAM control, the combination of T+SOX6, T+FOXQ1, and T+SOX6+FOXQ1 resulted in a strong significant increase in expression levels of ACAN and COL2, and a significant increase was also recognized in the expression level of CD24. As to PAX1, only the combination of T+FOXQ1 and T+SOX6+FOXQ1 made a significant difference, and T+SOX6 did not increase the expression level of PAX1. As to KRT8, only T+FOXQ1 made a significant difference, but the combination of the other two also showed a tendency of a strong increase in expression level.

Transduced MSCs cultured for three weeks in pellet cultures were similar to those identified in fibroblast cultures but showed more exaggerated characteristics. The SHAM control or T-transduced MSC pellet did not bring about identifiable Safranin-O-stained proteoglycans in the pellet, and vacuolated cells could not be identified therein. Transduction with T+SOX6+PITX1 or T+SOX6+PITX1+FOXQ1 showed some notochord cell-like cells, vacuoles, and a sporadic but small Safranin-O positive regions. T+SOX6-transduced cells showed a pellet filled with vacuolated cells together with potent but localized proteoglycan deposition. Finally, the combination of T+SOX6+FOXQ1 showed proteoglycan deposition distributed throughout the pellet. It was similar to extracellular matrix deposition derived from nucleus pulposus cells cultured in a pellet. Sporadically, vacuole-forming cells could be distinguished, but most part of cells showed a mature nucleus pulposus cell-like phenotype.

Finally, the expression levels of proteins of nucleus pulposus cell markers and notochord cell markers within MSC pellet cultures were checked by immunohistochemistry. A cryosection slide was rehydrated and blocked with 3% (v/v) BSA/PBS for 30 minutes. An intercellular protein section was blocked with 0.1% (w/v) Triton-X (FUJIFILM Wako Pure Chemical Corporation)/3% (v/v) BSA/PBS. Rabbit anti-COL2 antibody was used as a primary antibody for staining COL2. Likewise, mouse anti-CD24 antibody (Funakoshi Co., Ltd., Japan), rabbit anti-ACAN antibody (Millipore, USA), mouse anti-KRT18 antibody (Abcam plc., UK), goat anti-PAX1 antibody (Abcam plc.), and rabbit anti-PITX1 antibody were used. Finally, each section was stained with PE labeling antibody corresponding to the primary antibody and was mounted with VECTASHIELD mounting medium (Funakoshi Co., Ltd.) containing DAPI. Images were taken with a ZeissLSM 510 Meta Confocal Microscope (Zeiss, Germany).

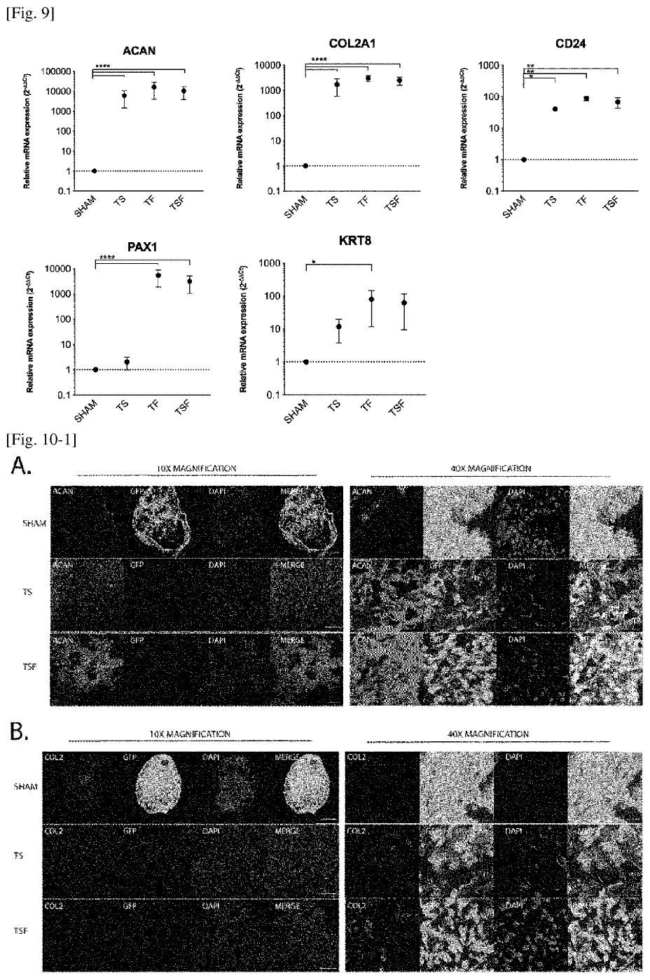

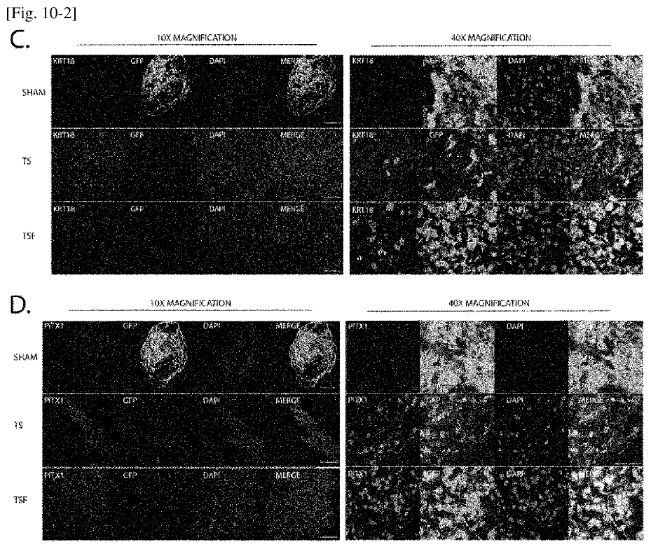

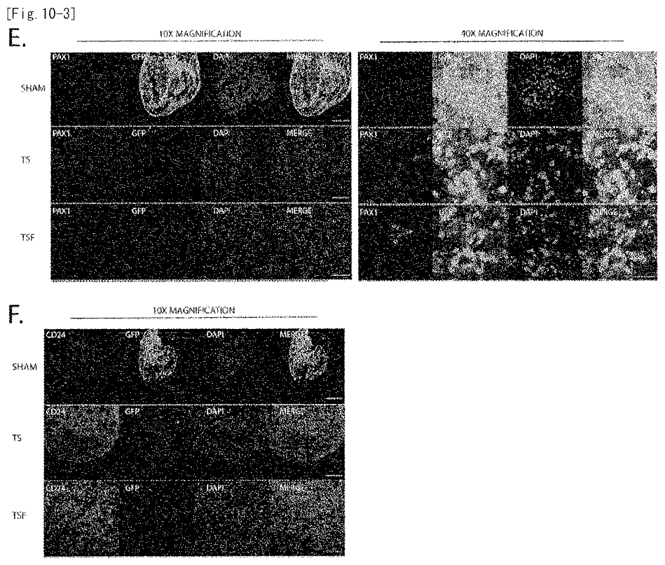

FIG. 10 shows the results. Pellets derived from MSCs transduced with T+SOX6 and T+SOX6+FOXQ1 showed a strong increase in nucleus pulposus cell marker expression as compared with the pellet of the SHAM control. As to ACAN, both T+SOX6 and T+SOX6+FOXQ1 showed a strong increase in expression as compared with the SHAM, but T+SOX6+FOXQ1 showed higher ACAN-positive expression than T+SOX6 (FIG. 10A). COL2 was demonstrated under all pellet conditions, but COL2 staining showed a particularly strong intensity under T+SOX6+FOXQ1 condition (FIG. 10B). KRT18 positivity was detected in both T+SOX6 and T+SOX6+FOXQ1, but no KRT18-positive cells were observed in the SHAM control (FIG. 10C). PITX1 showed a strong increase in positivity and intensity of T+SOX6 and T+SOX6+FOXQ1, as compared with the SHAM pellet, note that PITX1 was identified as an important marker transcription factor for a nucleus pulposus cell phenotype (FIG. 10D). Similarly, PAX1 was observed in T+SOX6 and T+SOX6+FOXQ1-transduced MSCs but was not observed under the SHAM condition (FIG. 10E). Finally, CD24 expression was strongly enhanced by transduction with T+SOX6 and T+SOX6+FOXQ1 (FIG. 10F). Overall, these findings are consistent with an increase in nucleus pulposus cell markers as observed in the mRNA expression analysis.

### (12) Consideration of Further Combinations of Transcription Factors

In the case where fibroblasts were transduced with T alone, no obvious increase was observed in expression levels of nucleus pulposus cell markers, but only the expression level of ACAN increased, as compared with the SHAM control. In contrast, in the case where fibroblasts were transduced with a combination of T and other transcription factors, a stronger and complete increase was observed in the expression levels of nucleus pulposus cell markers. For all three types of nucleus pulposus cell markers (ACAN,COL2, and CD24) tested, the expression levels with FOXQ1 combined with T were particularly excellent, and the expression levels strongly increased in the following order of SOX6, SOX9, HIF1α, FOXA2, PITX1, HIF3α, and RUNX1 when combined with T.

### [Reference Example] Evaluation of Optimal Combination of Growth Factors in Nucleus Pulposus Cell Induction Medium

Human bone marrow-derived MSCs (Lonza, Switzerland) were cultured in nucleus pulposus cell induction medium (NPIM) composed of DMEM high glucose (Dulbecco, USA) supplemented with 50 μM magnesium L-ascorbyl phosphate n-hydrate (FUJIFILM Wako Pure Chemical Corporation, Japan), 6.25 μg/mL insulin-transferrin-selenium-x (Thermo Fisher Scientific, Inc., USA) and 1% penicillin/streptomycin for two weeks. Further, 10 nM dexamethasone was added thereto. Concurrently, MSCs were separately suspended in 5 mL of NPIM and were centrifuged in a 15 mL propylene centrifugation tube for 5 minutes at 250 g. The cell cultures were incubated in a wet chamber at 37° C. under 21% O₂. Two days later, monolayer and cell pellet culture media containing TGFβ1, TGFβ2, or TGFβ3 (10 ng/ml) were further supplemented with GDF5 or GDF6 (100 ng/mL), and the cultures were transferred to a 5% O₂ wet chamber. The culture media were refreshed every two days. The monolayer cultures were harvested two weeks later by trypsinization with 0.25% EDTA/trypsin and lysed with a lysis solution (Ambion, USA) for real-time PCR analysis. The pellet cultures were harvested in the third week, supplemented with a Tissue-TEK O.C.T. compound (Sakura Finetek USA, Inc., USA), and snap-frozen in liquid nitrogen. Subsequently, fixed pellets were sliced into a 8 μm section using a cryostat. Each pellet was stained with a 1 g/l, safranin-O and 800 mg/L, fast green FCF staining solution.

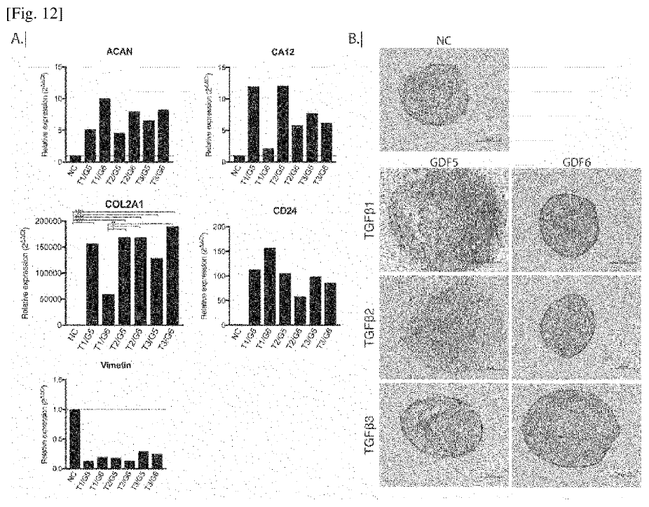

FIG. 12 shows the results. Addition of GDF5 or GDF6, and TGFβ-1, TGFβ-2 or TGFβ-3 showed a strong decrease in expression of vimentin, while showing strong expressions of aggrecan (ACAN), CA12, and CD24 in MSCs for all combinations of growth factors (FIG. 12A). Type II collagen (COL2A1) showed a significant increase in expressions excluding TGFβ1/GDF6 (which represents a combination of TGFβ1 and GDF6; the same applies to the following description). Further, TGFβ1/GDF5, TGFβ2/GDF5, TGFβ2/GDF6, and TGFβ3/GDF6 each showed a significant increase in expression of COL2A1 in MSCs, as compared with TGFβ1/GDF6. As to CA12, addition of TGFβ1/GDF5 and TGFβ2/GDF5 particularly showed a strong increase in expression level in the cultured cells, but addition of TGFβ1/GDF6 did not induce an increase in expression level.

Regarding the MSC pellet culture (FIG. 12B), a pellet supplemented with TGFβ1/GDF5 showed strong safranin-O staining for detecting proteoglycans (that is, strong deposition of proteoglycans), and a pellet supplemented with TGPβ2/GDF5 showed a slightly weaker safranin-O staining than above. In contrast, a culture supplemented with a combination of TGFβ3/GDP6 did not result in safranin-O staining, but TGPβ3/GDF5, TGF≈1/GDF6, and TGP≈2/GDF6 showed slight staining with safranin-O (that is, slight initial deposition of proteoglycans).

1. A differentiation inducer comprising an effective amount of a gene of nucleus pulposus cell master regulator transcription factors or a product thereof for use in differentiation induction of nucleated cells other than active nucleus pulposus cell phenotypes into an active nucleus pulposus cell phenotype, wherein
the master regulator transcription factors comprise:
Brachyury (T) or a homolog thereof; and
at least one selected from the group consisting of SRY-box6 (SOX6) or a homolog thereof and Forkhead Box Q1 (FOXQ1) or a homolog thereof.

2. The differentiation inducer according to claim 1, wherein the nucleus pulposus cell master regulator transcription factors further comprise at least one selected from the group consisting of Paired Like Homeodomain1 (PITX1) and Paired Box 1 (PAX1), or a homolog thereof.

3. The differentiation inducer according to claim 1, wherein the nucleus pulposus cell master regulator transcription factors further comprise at least one selected from the group consisting of Hypoxia inducible factor 3 alpha (HIF3α), SRY-box9 (SOX9), Runt-related Transcription Factor 1 (RUNX1), hypoxia Inducible Factor 1 alpha (HIF1α) and Forehead Box A2 (FOXA2), or a homolog thereof.

4. The differentiation inducer according claim 1, wherein the nucleus pulposus cell master regulator transcription factors are in the form of a gene inserted into an expression vector.

5. A pharmaceutical composition for use in treating or preventing an intervertebral disc disorder in a vertebrate animal, comprising the differentiation inducer according to claim 1.

6. A method for producing induced nucleus pulposus cells, comprising the steps of:
introducing the differentiation inducer according to claim 1 in vitro into nucleated cells other than active nucleus pulposus cell phenotypes (hereinafter referred to as “introduction step”); and performing transdifferentiation or differentiation induction into an active nucleus pulposus cell phenotype through culturing the transcription factor-introduced cells obtained by the introduction step (hereinafter referred to as “differentiation induction step”).

7. The method for producing induced nucleus pulposus cells according to claim 6, further comprising a step of checking an expression status of at least one selected from the group consisting of CD24, aggrecan, and type II collagen in the cells during culture or after culture in the differentiation induction step.

8. The method for producing induced nucleus pulposus cells according to claim 7, wherein the differentiation induction step comprises culturing the transcription factor introduction cells in a culture medium supplemented with transforming growth factor β1 (TG≈1) and growth differentiation factor 5 (GDF5).

9. The method for producing induced nucleus pulposus cells according to claim 7, wherein the differentiation induction step comprises culturing the transcription factor introduction cells under at least one condition selected from the group consisting of a hypoxic environment, an acidic environment, and a low-glucose environment.

10. Transcription factor-introduced cells that are nucleated cells other than active nucleus pulposus cell phenotypes comprising an effective amount of the nucleus pulposus cell master regulator transcription factors defined in claim 1.

11. Induced nucleus pulposus cells that are cells having an active nucleus pulposus cell phenotype obtained through culturing the transcription factor introduction cells according to claim 10.

12. The induced nucleus pulposus cells according to claim 11, wherein the induced nucleus pulposus cells are expressing at least one selected from the group consisting of CD24, aggrecan, and type II collagen.

13. The induced nucleus pulposus cells according to claim 11, wherein the induced nucleus pulposus cells are viable under at least one condition selected from the group consisting of a hypoxic environment, an acidic environment, and a low-glucose environment.

14. The induced nucleus pulposus cells according to claim 11, wherein the induced nucleus pulposus cells have intercellular vacuoles.

15. A cell population comprising the transcription factor-introduced cells according to claim 10.

16. A cell preparation for use in treating or preventing an intervertebral disc disorder in a vertebrate animal, comprising the cell population according to claim 11.

17. A method for treating or preventing an intervertebral disc disorder in a vertebrate animal, comprising transplanting or administering the induced nucleus pulposus cells according to claim 11 in vivo so as to act on the intervertebral disc nucleus pulposus tissue.

18. A method for treating or preventing an intervertebral disc disorder in a vertebrate animal, comprising administering the differentiation inducer according to claim 1 in vivo so as to act on nucleus pulposus cells in an intervertebral disc.

19. A method for screening for a medicine or a method for treating or preventing an intervertebral disc disorder in a vertebrate animal, comprising a step of testing effectiveness and safety in a subject using the transcription factor-introduced cells according to claim 10.

20. A method for obtaining an indicator associated with an aging, degenerating or disease state of nucleus pulposus cells, comprising measuring expression levels of the nucleus pulposus cell master regulator transcription factors defined in claim 1 in isolated nucleus pulposus cell.

21. The cell population according to claim 11, further comprising induced nucleus pulposus cells having an active nucleus pulposus cell phenotype obtained through culturing the transcription factor-induced cells.
